SEQUENCE LISTING (23 sequences)

SEQ ID NO: 1 - DNA, 20 bp, Artificial Sequence
  BDNF IV Forward primer
gtgaggtttgtgtggacccc

SEQ ID NO: 2 - DNA, 20 bp, Artificial Sequence
  BDNF IV Reverse primer
attgggccgaactttctggt

SEQ ID NO: 3 - DNA, 19 bp, Artificial Sequence
  CREBa Forward primer
ggctccagattccatggtc

SEQ ID NO: 4 - DNA, 20 bp, Artificial Sequence
  CREBa Reverse primer
gtgttacgtgggggagagaa

SEQ ID NO: 5 - DNA, 19 bp, Artificial Sequence
  CREBb Forward primer
ggctccagattccatggtc

SEQ ID NO: 6 - DNA, 19 bp, Artificial Sequence
  CREBb Reverse primer
ggctccagattccatggtc

SEQ ID NO: 7 - DNA, 24 bp, Artificial Sequence
  GAPDH Forward primer
tggtctcctctgacttcaacagcg

SEQ ID NO: 8 - DNA, 24 bp, Artificial Sequence
  GAPDH Reverse primer
aggggtctacatggcaactgtgag

SEQ ID NO: 9 - DNA, 22 bp, Artificial Sequence
  BDNF I Forward primer
ttgaagctttgcggatattgcg

SEQ ID NO: 10 - DNA, 20 bp, Artificial Sequence
  BDNF I Reverse primer
aagttgccttgtccgtggac

SEQ ID NO: 11 - DNA, 26 bp, Artificial Sequence
  BDNF II Forward primer
tggtatactgggttaactttgggaaa

SEQ ID NO: 12 - DNA, 20 bp, Artificial Sequence
  BDNF II Reverse primer
aagttgccttgtccgtggac

SEQ ID NO: 13 - DNA, 29 bp, Artificial Sequence
  BDNF IV Forward primer #2
gaaatatatagtaagagtctagaaccttg

SEQ ID NO: 14 - DNA, 20 bp, Artificial Sequence
  BDNF IV Reverse primer #2
aagttgccttgtccgtggac

SEQ ID NO: 15 - DNA, 22 bp, Artificial Sequence
  BDNF VI Forward primer
gctttgtgtggaccctgagttc

SEQ ID NO: 16 - DNA, 20 bp, Artificial Sequence
  BDNF VI Reverse primer
aagttgccttgtccgtggac

SEQ ID NO: 17 - DNA, 24 bp, Artificial Sequence
  BDNF VII Forward primer
cctgaaagggtctgcggaactcca

SEQ ID NO: 18 - DNA, 24 bp, Artificial Sequence
  BDNF VII Reverse primer
gaagtgtacaagtccgcgtcctta

SEQ ID NO: 19 - DNA, 25 bp, Artificial Sequence
  BDNF IX Forward primer
ggactatgctgctgacttgaaagga

SEQ ID NO: 20 - DNA, 24 bp, Artificial Sequence
  BDNF IX Reverse primer
gagtaaacggtttctaagcaagtg

SEQ ID NO: 21 - DNA, 22 bp, Artificial Sequence
  GAPDH Forward primer #2
atggtgaaggtcggtgtgaacg

SEQ ID NO: 22 - DNA, 22 bp, Artificial Sequence
  GAPDH Reverse primer #2
cgctcctggaagatggtgatgg

SEQ ID NO: 23 - protein, 11 aa, Human immunodeficiency virus
YGRKKRRQRRR

(19) United States
(12) Patent Application Publication
(10) Pub. No.: US 2024/0197760 A1
(43) Pub. Date: Jun. 20, 2024

(54) ACTIVATORS OF INTEGRATED STRESS RESPONSE PATHWAY FOR PROTECTION AGAINST FERROPTOSIS

(71) Applicant: Burke Neurological Institute, White Plains, NY (US)

(72) Inventor: Rajiv R. Ratan, White Plains, NY (US)

(21) Appl. No.: 18/285,815

(22) PCT Filed: Apr. 8, 2022
(86) PCT No.: PCT/US2022/023986

§ 371 Date: Oct. 5, 2023

Related U.S. Application Data
(60) Provisional application No. 63/172,927, filed on Apr. 9, 2021.

(51) Int. Cl. A61K 31/7004; A61P 25/28

(52) U.S. Cl. CPC A61K 31/7004; A61P 25/28

(57) ABSTRACT

Methods, agents, and pharmaceutical compositions are provided for treating neurodegenerative disease, for enhancing stroke recovery, and or for inhibiting ferroptosis in a subject. These agents, including 2-deoxyglucose, can be used alone or with the right timing in sequential combination with agents that inhibit the integrated stress response. The present invention addresses these needs and provides methods of treating neurodegenerative conditions, including without the need for dietary restrictions which can be challenging in already unwell patients.

### CROSS-REFERENCE TO RELATED APPLICATIONS

This application claims benefit of U.S. Provisional Application No. 63/172,927, filed Apr. 9, 2021, the contents of which are hereby incorporated by reference.

### BACKGROUND OF THE INVENTION

The disclosures of all publications, patents, patent application publications and books referred to in this application are hereby incorporated by reference in their entirety into the subject application to more fully describe the art to which the subject invention pertains.

Recent studies have shown that brain aging and age-associated neurological diseases such as Alzheimer's disease may begin decades before their symptoms are manifest. Accordingly, identifying dietary manipulations that could forestall these slowly-evolving changes could provide widely applicable, practical ways to limit the growing epidemic of disability from age associated neurological conditions.

Among many options, intermittent fasting (IF) is a dietary intervention widely implicated in brain health, cognitive aging, Alzheimer's disease and stroke. During the fasting phase of IF, glycogen stores are depleted leading to low circulating glucose levels and a metabolic switch whereby adipose cells release fatty acids to the liver where they are converted to ketone bodies.

Reducing glucose and increasing ketone bodies via IF dietary intervention has been shown to be associated with improvements in a host of preclinical models of brain physiology and pathology.

Alzheimer's disease (AD) is the most prevalent form of age-associated dementia, characterized by a number pathophysiological changes such as extracellular deposition of amyloid-beta (Aβ) plaques, formation of neurofibrillary tangles, inflammation, synaptic loss, and neuronal death along with cognitive deficit (Grontvedt et al., 2018). Several clinical trials are underway targeting these pathological hallmarks of the disease but without much success in improving cognitive function. Recently, the United States Food and Drug Administration approved (US FDA) approved an anti-amyloid immunotherapy, aducanumab, to slow the progression of the disease but the approval was quite controversial with respect to improving the clinical recovery in AD patients. This underlines the necessity for alternative strategies focused on not only removing these pathological markers of the disease but also improving the clinical recovery including cognitive function and associated psychiatric disturbances in AD patients.

The previous animal studies and human population studies have found several beneficial effects including cognitive improvement with diet restriction regimens such as calorie restriction (CR) and intermittent fasting (IF). The ENCORE study (Exercise and Nutrition Interventions for Cardiovascular Health) found that the combination of exercise and calorie restriction improves neurocognitive function (Smith et al., 2010). Another prospective intervention trial showed significant improvement in memory in healthy elderly humans with calorie restriction (30% CR) for three months (Witte et al., 2009). In a recent clinical trial, CR induced weight loss was found to be strongly associated with cognitive improvement in obese elderly patients with mil cognitive impairment (MCI) (Horie et al., 2016). A more recent multicenter randomized controlled trial in healthy non-obese adults implementing CR regimen for two years showed a significant improvement in working memory (Leclerc et al., 2020). In a recent small randomized controlled trial, even ketogenic formula that mimics the physiological state of CR, when given for 12 weeks to 20 patients with mild-moderate AD led to a significant improvement in working memory, short term memory, and processing speed (Ota et al., 2019). A recent three-year follow-up study in an elderly population with MCI has also shown significant improvement in cognitive function with IF regimen (Ooi et al., 2020). Many preclinical studies with calorie restriction have also found significant improvement in cognitive or motor function in animal models of AD and other chronic and acute neurological disorders (Dias et al., 2020; Gudden et al., 2021; Halagappa et al., 2007; Liu et al., 2019; Roberge et al., 2008; Rubovitch et al., 2019).

The mechanism is still not clear, but many recent studies have helped uncover the possible underlying mechanisms of the beneficial effects of these dietary restriction regimens which has been beautifully reviewed by Mark Mattson elsewhere (de Cabo and Mattson, 2019; Mattson et al., 2018). There are indications that the beneficial effects of dietary restriction are related with the homeostatic responses such as improvement in glucose metabolism, increase in stress resilience, and suppression of inflammation (de Cabo and Mattson, 2019). One of the major limitations associated with these dietary restriction regimens is the difficulty in their implementation as therapeutic options. There are several drugs in development that could achieve the benefits of these regimens without the need for diet restriction. 2-Deoxyglucose (2-DG) is one such mimetic that has been shown to mimic intermittent fasting (Duan and Mattson, 1999; Wan et al., 2004) and also to improve brain energy metabolism and amyloid pathology in triple transgenic model of AD (Yao et al., 2011). However, the consequences of 2-DG treatment on cognitive function in AD or sensory motor function in stroke along with the underlying mechanism were not known. Additionally, the mechanism of 2-DG action at cellular level is based on its function as a glycolytic inhibitor. But it is not clear how 2-DG led changes in glucose metabolism could be sensed by plasticity genes involved in learning and memory and repair programs.

The present invention addresses these needs and provides methods of treating neurodegenerative conditions, including without the need for dietary restrictions which can be challenging in already unwell patients.

### SUMMARY OF THE INVENTION

A method of protecting a cell in a subject against ferroptosis comprising administering to the subject an amount of an activator of an Integrated Stress Response pathway effective to increase phosphorylation of a Eukaryotic Initiation Factor 2 alpha (eif2alpha), or to elicit Bdnf gene expression, and thereby reduce ferroptosis.

A method of protecting a cell in a subject against ferroptosis, wherein the subject has, or is experiencing, a neurodegenerative disease comprising administering to the subject an amount of an activator of an Integrated Stress Response pathway effective to increase phosphorylation of an eif2alpha, or to elicit Bdnf gene expression, and thereby reduce ferroptosis.

A method of treating a subject for a stroke, Parkinson's disease, Alzheimer's disease, epilepsy, or Huntington's disease, comprising administering to the subject an amount of an activator of an Integrated Stress Response pathway effective to increase phosphorylation of an eif2alpha so as to treat the stroke, Parkinson's disease, Alzheimer's disease, epilepsy, or Huntington's disease.

A method of treating a neurodegenerative disease in a subject comprising administering to the subject an amount of an activator of 2-deoxyglucose effective to treat a neurodegenerative disease.

A pharmaceutical composition comprising a pharmaceutically acceptable carrier and an activator of an Integrated Stress Response pathway effective to increase phosphorylation of a Eukaryotic Initiation Factor 2 alpha (eif2alpha) for reducing ferroptosis.

A pharmaceutical composition comprising a pharmaceutically acceptable carrier and an activator of an Integrated Stress Response pathway effective to increase phosphorylation of a Eukaryotic Initiation Factor 2 alpha (eif2alpha) for enhancing gene expression associated with learning, memory and/or plasticity in the human CNS.

A pharmaceutical composition comprising a pharmaceutically acceptable carrier and an inhibitor of N-linked glycosylation for promoting plasticity and/or adaptation to injury in the human CNS.

A method of treating a neurodegenerative disease in a subject comprising administering to the subject an amount of an inhibitor of N-linked thereby treating the neurodegenerative disease.

A method of treating a subject for a cancer comprising administering to the subject an amount of an activator of an Integrated Stress Response pathway effective to increase phosphorylation of an eif2alpha and thereby reduce ferroptosis in the subject so as to treat the cancer.

A method of protecting a cell in a plant from heat stress comprising treating the plant with an amount of an activator of an Integrated Stress Response pathway effective to increase phosphorylation of an eif2alpha and thereby reduce ferroptosis in the plant and protect from heat stress.

### BRIEF DESCRIPTION OF THE FIGURES

FIG. 1A-1L: Decreasing neuronal glucose uptake and utilization induces plasticity gene program and LTP. (1A) Live (Green)-dead (Red) (Calcein-AM and ethidium homodimer staining) staining of mouse primary neurons treated with 2-DG (1 mM-15 mM) for 24 h. (Scale bar 50 μm. n=3 independent cultures) (1B-1D) Changes in mRNA levels of Creb1, Bdnf, and Rbbp4 in response to increasing concentrations of 2-DG treated for 6 h in mouse primary neurons. (n=3, One-way ANOVA with Dunnett post-hoc test; * p<0.05; ** p<0.01; and *** p<0.001) (1E-1G) Changes in mRNA levels of Creba, Crebb, and Bdnf exon IV in response to increasing concentrations of 2-DG treated for 6 h in human i.p.s. derived mature cortical neurons. (n=3, One-way ANOVA with Dunnett post-hoc test; *p<0.05; ** p<0.01; and *** p<0.001) (1H) Change in Bdnf gene expression in the mouse brain cortex in response to 2-DG (10 mg/Kg) intra-peritoneal injection for 6 h. (n=12, Student's t-test) (1I) Validation of wild type and Glut3 heterozygous knock out mice brain cortex tissues for Glut3 protein using anti-Glut3 antibody. (1J) The mRNA level of Bdnf in brain cortices of three months old wild type (WT) and Glut3 heterozygous knock out mice (Glut3+/−). (n=7, Student's t-test) (1K) Change in mature Bdnf protein in response to 2-DG (10 mM) treatment for 6 h in mouse immature primary neurons. (n=3, Student's t-test) (1L) Change in the level of fEPSP in hippocampal slices submerged either in ACSF alone (Vehicle) or in ACSF with 10 mM 2-DG for 6 h. (Two-way ANOVA with repeated measures revealed significant difference between two groups (F(1, 26)=7.327, p=0.0118).

FIG. 2A—21: 2-DG normalizes learning and memory deficit in 5×FAD mice. (2A) A schematic diagram showing the experimental set up of the Alzet (osmotic) pump filled with either saline or 2-DG implanted below the neck of mice and connected with its catheter tube to the brain cortex through intra-cerebro-ventricular (ICV) injection. Six-month-old wild type and 5×FAD mice had Alzet pump implanted in their brain cortices for four weeks allowing slow release of either saline or 2-DG and then their memory related behaviors were assessed followed by LTP study in hippocampal slices and Bdnf gene expression studies in cortex tissues (2B) Change in the mRNA level of Bdnf in brain cortices of wild type and 5×FAD (n=10 for each group, One-way ANOVA with Turkey's multiple comparison test). (2C) Changes in the level of fEPSPs in hippocampal slices dissected out from wild type and 5×FAD mice. Two-way ANOVA with repeated measures showed significant effects for groups (F(3, 40)=3.252, p=0.0316) and time (F(60, 2400)=5.261, p<0.0001) but not for interaction between groups and time (F(180, 2400)=0.8259, p=0.9527). Tukey's multiple comparison revealed significant difference between 5×FAD (saline) and 5×FAD (2-DG) (p=0.0194) (2D) A schematic diagram showing experimental set up of spontaneous alteration (Y-maze) behavior (To assess short term memory). (2E) Changes in spontaneous alteration behaviors in wild type and 5×FAD mice (n=9 for each group, One-way ANOVA with Turkey's multiple comparison test). (2F) A schematic diagram showing experimental set up of Morris water maze behavior (To assess long term memory). (2G) Seven-day spatial reference memory training to reach the island (hidden platform) by wild type and 5×FAD mice (n=9 for each group). Two-way ANOVA with repeated measures showed significant effects for days (F(4.325, 138.4)=71.19, p<0.0001) but not for groups (F(3, 32)=2.592, p=0.0698) and interaction (F(18, 192)=0.1013, p=0.0316). Analysis of escape latency on each day by Fisher's LSD post-hoc test revealed that 5×FAD mice injected with 2-DG showed significant difference from 5×FAD mice injected with saline on days 5 and 7 (Day 5, p=0.0343; Day 7, p=0.0233). (2H) Probe trial of the wild type and 5×FAD mice on day 8 (n=9 for each group, One-way ANOVA with Turkey's multiple comparison test). (2I) A representative summary of the findings showing that 2-DG significantly induces plasticity gene program, enhances long term potentiation, and restores short term and long-term memory in 5×FAD mice.

FIG. 3A-3G: Post-stroke 2-DG treatment improves sensory and motor functional recovery. (3A) A schematic diagram showing the experimental set up of inducing transient ischemic stroke with 60 min MCAO in the mouse followed by post-ischemic intra-peritoneal (i.p.) injections of 2-DG (10 mg/Kg) and behavioral assessment at various time intervals. (3B) Corner test. Two-way ANOVA with repeated measures showed significant effects for days (F(3.378, 121.6)=49, p<0.001), groups (F(3, 36)=110.1, p<0.001), and interaction between days and groups (F(15, 180)=14.24, p<0.001). Analysis of behavior in corner test at various time intervals by Fisher's LSD post-hoc test revealed that MCAO mice injected with 2-DG showed significant difference from MCAO mice injected with saline on days 21 and 28 (Day 21, p=0.0221; Day 28, p=0.0386). (3C) Tape removal test. Two-way ANOVA with repeated measures showed significant effects for days (F(4.261, 153.4)=16.97, p<0.0001), groups (F(3, 36)=47.9, p<0.0001), and interaction between days and groups (F(15, 180)=5.705, p<0.0001). Analysis of behavior in tape removal task at various time intervals by Fisher's LSD post-hoc test revealed that MCAO mice injected with 2-DG showed significant difference from MCAO mice injected with saline on days 21 and 28 (Day 21, p=0.0235; Day 28, p=0.0219). (3D) Pole test. Two-way ANOVA with repeated measures showed significant effects for days (F(2.638, 94.99)=21.03, p<0.0001), groups (F(3, 36)=13.68, p<0.0001), and interaction between days and groups (F(15, 180)=5.777, p<0.0001). Analysis of behavior in pole test at various time intervals by Fisher's LSD post-hoc test revealed that MCAO mice injected with 2-DG showed significant difference from MCAO mice injected with saline on day 21 (Day 21, p=0.0037). (3E) A schematic diagram showing the experimental set up of inducing hemorrhagic stroke with intra-cranial injection of collagenase in striatum of the mouse followed by post-ischemic intra-peritoneal (i.p.) injections of 2-DG (10 mg/Kg) and behavioral assessment at various time intervals. (3F) Tape removal test. Two-way ANOVA with repeated measures showed significant effects for days (F(4.447, 160.1)=24.62, p<0.0001), groups (F(3, 36)=116.7, p<0.0001), and interaction between days and groups (F(15, 180)=7.997, p<0.0001). Analysis of behavior in tape removal task at various time intervals by Fisher's LSD post-hoc test revealed that ICH mice injected with 2-DG showed significant difference from ICH mice injected with saline on days 7 and 21 (Day 7, p=0.0116; Day 21, p=0.0135). (3G) A schematic summary of the findings indicating that 2-DG improves sensory-motor function in mice models of both ischemic and hemorrhagic stroke.

FIG. 4A-4H: 2-DG led induction of plasticity program is not mediated by AMPK mediated energy sensing. (4A) Relative changes in AMP/ATP ratio calculated from peak intensities obtained from non-targeted metabolic profiling study of mouse primary neurons treated with 10 mM 2-DG for 8 h. (n=3, Student's t-test) (4B) Relative changes in the ratio of p-AMPK and total AMPK protein in mouse primary neurons treated with different concentrations of 2-DG for 4 h. Metformin (5 mM) was used as a positive control. (n=3, One-way ANOVA with Dunnett post-hoc test) (4C) Changes in mRNA levels of Bdnf in response to increasing concentrations of 2-DG in mouse immature primary neurons. (n=3, One-way ANOVA with Dunnett post-test). (4D) A schematic diagram depicting the dissociation between AMPK phosphorylation and plasticity gene program expression (BDNF was used as a surrogate marker of the plasticity gene program). (4E) Verification of the knockdown of AMPK phosphorylation with the overexpression of the adenoviral construct of the dominant negative AMPK using p-AMPK and total AMPK western blots. (n=3) (4F, 4G) Changes in mRNA levels of Creb1 and Bdnf in response to increasing concentrations of 2-DG in mouse immature primary neurons overexpressing either GFP or dominant negative AMPK (AMPK D.N.). (n=3, Two-way ANOVA with Bonferroni post-hoc test). (4H) A schematic summary of the findings indicating that 2-DG led increase in plasticity gene program is not mediated by AMPK phosphorylation.

FIG. 5A-5F: 2-DG led induction of plasticity program is transcription dependent. (5A-5C) Changes in mRNA levels of Creb1, Bdnf, and Rbbp4 in response to 6 h treatment of 2-DG or 2-DG in combination with transcriptional inhibitor, actinomycin D (ActD) in mouse immature primary neurons. (n=3, One-way ANOVA with Bonferroni post-hoc test) (5D) The change in mRNA level of Bdnf in hippocampal slices submerged in artificial cerebrospinal fluid (ACSF) with either saline, 2-DG (10 mM) or 2-DG (10 mM) plus transcriptional inhibitor, actinomycin D (ActD, 1 μg/ml). (n=3, One-way ANOVA with Bonferroni post-hoc test) (5E) Change in the level of fEPSP in hippocampal slices submerged in ACSF with vehicle, 2-DG (10 mM), ActD (1 μg/ml) or 2-DG (10 mM) and ActD (1 μg/ml) for 6 h. Two-way ANOVA with repeated measures showed significant effects for groups (F(3, 50)=4.960, p=0.0043), time (F(60, 3000)=14.43, p<0.0001) and interaction between groups and time (F(180, 3000)=1.822, p<0.0001). Tukey's multiple comparison revealed significant difference between vehicle and 2-DG (p=0.0133) but no significant difference between ActD and 2-DG plus ActD (p=0.8915) (5F) A schematic summary demonstrating that 2-DG led increase in plasticity gene program is transcription dependent.

FIG. 6A-6H: High throughput RNA sequencing reveals the dominant gene signatures related to unfolded protein response. (6A) A heat map showing dose dependent changes in scaled expression of plasticity genes in response to 1 mM and 10 mM 2-DG treatments for 6 h in mouse primary neurons. (6B) Gene set enrichment analysis (GSEA) indicating changes in hallmarks of gene sets in response to 1 mM and 10 mM 2-DG treatments for 6 h in mouse primary neurons. Values are normalized enrichment scores. Normalized enrichment score (NES)>2 is considered as significant. (6C) The bar chart representation of Gene Ontology (GO) analysis of cellular component enrichment. The FDR (False discovery rate) values are indicated in log scale. (6D) Differential expression of genes related to unfolded protein response (UPR) in response to 1 mM and 10 mM 2-DG treatments for 6 h. (6E) Increased eIF2a phosphorylation with 2-DG (10 mM) treatment for 2 h in mouse primary neurons. (n=3) (6F, 6G) Increase in expression of ATF4 target genes, Trib3 and Chac1 with 2-DG treatment for 6 h in mouse primary neurons. (n=3, Student t test) (6H) A schematic summary of findings indicating endoplasmic reticulum associated unfolded protein response as the dominant effector of 2-DG led decrease in glucose utilization.

FIG. 7A-7R: ATF4 mediates the 2-DG led induction of plasticity gene program. (7A, 7B) Change in mRNA level of Bdnf with increasing concentrations of D-glucose or D-mannose either co-treated with 2-DG or without co-treatment for 6 h in mouse primary neurons. (n=3, Two-way ANOVA with Bonferroni post-hoc test) (7C) Western blot and densitometric analyses of three independent sets showing an increase in eIF2a phosphorylation with tunicamycin (3 μM) treatment for 2 h in mouse primary neurons. (n=3, Student's t test) (7D-7F) Increase in mRNA levels of Bip, Creb1, and Bdnf with tunicamycin (3 μM) treatment for 6 h in mouse primary neurons. (n=3, Student's t test) (7G) Western blot showing that 2 h co-treatment of PERK inhibitor I (5 μM) (GSK2606414) blocks 2-DG (10 Mm) and thapsigargin (500 nM) led increase in eIF2 phosphorylation in mouse primary neurons. (n=3) (7H-7K) PERK inhibitor I (5 μM) when co-treated with 2-DG (10 mM) for 9 h blocked 2-DG led increase in levels of Trib3 promoter reporter activity, Trib3 mRNA, Bdnf promoter reporter activity, and Bdnf mRNA, respectively. 9 h time period was chosen for this experiment as the time course for Trib3 promoter activity with 2-DG treatment showed Trib3 promoter activity to be in good dynamic range (n=3, Two-way ANOVA with Bonferroni post-hoc test) (7L, 7M) Increase in levels of Bdnf mRNA and LTP, respectively, in hippocampal slices dissected out from the homozygous floxed Ppp1r15b mice with transient knockdown of Ppp1r15b in their hippocampi through intracranial injection of AAV8-Cre. AAV8-GFP was injected as a viral control. (n=17, Student's t test for analyzing changes in the level of Bdnf mRNA and two-way ANOVA test with repeated measures for analyzing the LTP data). Two-way ANOVA with repeated measures revealed significant difference between two groups (F(1, 26)=13.12, p=0.0012) (7N) Mutation in ATF4 binding site blocked 2-DG (9 h treatment) induced activity of Trib3 promoter reporter transiently expressed in mouse primary neurons for 24 h. (n=3, Two-way ANOVA with Bonferroni post-hoc test) (7O) Transient overexpression of adenoviral construct of dominant negative ATF4 for 48 h (with six amino acid substitutions within the DNA binding domain as described before) occluded 2-DG (6 h treatment) led increase in Bdnf gene expression. (n=3, Two-way ANOVA with Bonferroni post-hoc test) (7P) Transient overexpression of adenoviral construct of ATF4 for 24 h led to significant increase in Bdnf gene expression. (n=3, Student t test) (7Q) Quantitative luciferase activity measurement (From pseudocolored bioluminescence) showed that 2-DG intraperitoneal injection (10 mg/Kg) for 4 h leads to paradoxical increase in the ATF4 translation in the mouse brain. CMV 5′UTR ATF4 luciferase reporter cloned into AAV8 viral vector was injected intracranially into the mouse brain and was allowed to express for three weeks before 2-DG i.p. injection. (Student t test) (7R) A schematic diagram showing UPR and its downstream target ATF4 mediates 2-DG led increase in the expression of Bdnf gene.

FIG. 8A-8H: ATF4 directly regulates Bdnf and other plasticity genes. (8A) Changes in endogenous mRNA levels of different Bdnf transcript variants in response to 6 h treatment of 10 mM 2-DG in primary neurons. (n=3, Student's t test) (8B) Schematic of different human Bdnf transcript variants (8C) Changes in promoter-reporter activities of different Bdnf transcript variants in response to 6 h treatment of 10 mM 2-DG in HT22 cells. (n=3, Student's t test) (8D, 8E) UCSC Genome Browser views of representative ChIP-Seq showing specific increase in binding of ATF4 at Bdnf close to transcription start site in response to 2-DG treatment (10 mM) for 6 h in primary neurons. Peak calls for each IP sample were adjusted for background by using those of Input samples for each condition (Control or 2-DG). (n=5) (8F) De novo motif analysis in the ATF4 differential binding peaks to identify the most enriched sequences within the peak regions. The analysis returned ATF4 as the most significant motifs, validating the ChIP specificity. (n=5) (8G) Volcano plot of differential peak calling of various plasticity genes in response to 2-DG (10 mM) for 6 h in primary neurons analyzed using DiffBind (DESeq2). Peak calls for each IP sample were adjusted for background by using those of Input samples for each condition (Control or 2-DG). (n=5). (8H) A schematic diagram showing that 2-DG leads to upregulation of Bdnf gene expression through direct binding of ATF4 at BDNF which dimerizes through an unknown binding partner.

FIG. 9A-9B: Non-targeted Metabolomics identifies significantly enriched and/or affected metabolic pathways in response to 2-DG treatment. Primary neurons were treated with 10 mM 2-DG for 24 h and then cells were harvested for non-targeted metabolomics study. (9A) Quantitative Enrichment Analysis (QEA) of different metabolite sets showing significantly enriched metabolic pathways. The data was obtained using QEA method of the metabolite set enrichment analysis (MSEA) tool of MetaboAnalyst. Pathways are depicted in the order of decreasing significance from top to bottom (increasing nominal p values, colored from red to yellow) with bars indicating their estimated fold enrichment. (9B) Pathway impact analysis of significantly altered metabolites showing the most impacted pathways (impact scores calculated from the topological analysis and indicated in X-axis) with an Impact score>0.1 as identified by metabolites showing significant pathway enrichment with a corresponding p value<0.05 (i.e., −log(p)>2.99 indicated on Y-axis). The quantitative enrichment tests (Global-test and Global-Ancova) was performed using MetaboAnalyst.

FIG. 10A-10C: Decreasing neuronal glucose uptake and utilization with the glycolytic inhibitor, glucosamine, induces plasticity gene program. (10A-10C) Changes in mRNA levels of Creb1, Bdnf, and Rbbp4 in response to increasing concentrations of a glycolytic inhibitor, glucosamine, treated for 6 h in mouse immature primary neurons. (n=3, One-way ANOVA with Dunnett post-hoc test; * p<0.05; ** p<0.01; and *** p<0.001)

FIG. 11A-11B: 2-DG treatment leads to a dose dependent increase in plasticity gene program in depolarized primary neurons. (11A, 11B) Neurons were treated with the increasing concentration of either 2-DG alone or co-treated with 25 mM KCl (to depolarize neurons) or co-treated with 25 Mm NaCl (as an osmolarity control for KCl) for 6 h and, thereafter, cells were processed for RNA extraction and gene expression study. (n=3, One-way ANOVA with Dunnett post-hoc test; * p<0.05; ** p<0.01; and *** p<0.001)

FIG. 12A-12C: Glucosamine led induction in plasticity gene program is transcription dependent. (12A-12C) Changes in mRNA levels of Creb1, Bdnf, and Rbbp4 in response to 6 h treatment of glucosamine or glucosamine in combination with transcriptional inhibitor, actinomycin D (ActD) in mouse immature primary neurons. (n=3, One-way ANOVA with Bonferroni post-hoc test)

FIG. 13A-13C: High throughput RNA sequencing indicating activation of UPR enabling upregulation of ATF4 as the master transcriptional regulator. (13A) Gene set enrichment analysis (GSEA) showing fold enrichment of dominant gene signatures in response to 6 h treatment of 2-DG (1 mM and 10 mM) in primary neurons. Values are normalized enrichment scores. Normalized enrichment score (NES)>2 is considered as significant. (13B) The bar chart representation of Gene Ontology (GO) analysis of cellular component enrichment in response to 1 mM 2-DG treatment. The FDR (False discovery rate) values in indicated in log scale. (13C) Ingenuity pathway analysis (Upstream regulator analysis) shows ATF4 is a major upstream regulator in response to 2-DG treatment.

FIG. 14A-14B: PERK signaling is necessary for glucosamine led increase in Bdnf gene expression. (14A) PERK inhibitor I (GSK2606414) completely blocked glucosamine induced expression of ATF4 target gene, Trib3. (14B) Same concentration of PERK inhibitor 1 also blocked glucosamine led increase in Bdnf gene expression. (n=3, Two-way ANOVA with Bonferroni post-hoc test)

FIG. 15A-15C: Validation of the activation of eIF2a signaling with the transient knockdown of Ppp1r15b in the hippocampal slices from homozygous floxed Ppp1r15b mice. Increase in the expression of ATF4 target genes such as Trib3, Chac1, and Ddit3 in hippocampal slices, which are activated downstream of the activation of eIF2a signaling. The hippocampal slices were dissected out from the homozygous floxed Ppp1r15b mice with transient knockdown of Ppp1r15b in their hippocampi through intracranial injection of AAV8-Cre (The viral expression was allowed for 3 weeks before the dissection). AAV8-GFP was injected as a viral control. (n=17, Student's t test)

FIG. 16A-16B: Validation of the activation of ATF4 signaling with the transient overexpression of adenoviral construct of ATF4 in mouse primary neurons. (16A, 16B) Increase in the expression of ATF4 target genes, Trib3 and Chac1 24 h after the transduction with adenoviral constructs of GFP or ATF4 in mouse primary neurons. (n=3, Student's t test)

FIG. 17: Art schematic of pathways involved in N-linked glycosylation in endoplasmic reticulum and cytosol. In view of the results disclosed herein, inhibiting any of these will be therapeutic for neurodegenerative conditions and stroke.

### DETAILED DESCRIPTION OF THE INVENTION

A method of protecting a cell in a subject against ferroptosis comprising administering to the subject an amount of an activator of an Integrated Stress Response pathway effective to increase phosphorylation of an Eukaryotic Initiation Factor 2 alpha (eif2alpha), or to elicit Bdnf gene expression, and thereby reduce ferroptosis.

A method of protecting a cell in a subject against ferroptosis, wherein the subject has, or is experiencing, a neurodegenerative disease comprising administering to the subject an amount of an activator of an Integrated Stress Response pathway effective to increase phosphorylation of an eif2alpha, or to elicit Bdnf gene expression, and thereby reduce ferroptosis.

In embodiments, the neurodegenerative disease is stroke, Parkinson's disease, Alzheimer's disease, epilepsy, or Huntington's disease.

A method of treating a subject for a stroke, Parkinson's disease, Alzheimer's disease, epilepsy, or Huntington's disease, comprising administering to the subject an amount of an activator of an Integrated Stress Response pathway effective to increase phosphorylation of an eif2alpha so as to treat the stroke, Parkinson's disease, Alzheimer's disease, epilepsy, or Huntington's disease.

In embodiments, the method increases phosphorylation of an eif2alpha and reduces ferroptosis.

In embodiments, the methods further comprise administering an anti-ferroptotic agent to the subject.

In embodiments, the anti-ferroptotic agent is a system xc-inhibitor, a GPX4 inhibitor, or a compound that indirectly inhibitsGPX4 activity by GSH depletion.

In embodiments, the anti-ferroptotic agent is rifampicin, promethazine, omeprazole, indole-3-carbinol, carvedilol, propranolol, estradiol, or a thyroid hormone.

In embodiments, the system xc-inhibitor is erastin, sulfasalazine, or sorafenib.

In embodiments, the method treats Alzheimer's disease.

In embodiments, the subject has had, or is experiencing, a stroke and wherein the method enhances stroke recovery in the subject.

In embodiments, the activator of Integrated Response Pathway is 2-deoxyglucose or (4R,5S,6R)-6-(hydroxymethyl)oxane-2,4,5-triol.

In embodiments, the subject (i) does not have a cancer, (ii) has not been diagnosed with a cancer, and/or (iii) has not been treated for a cancer.

In embodiments, the subject (i) is not on a calorie-restricted diet regime, (ii) has not been prescribed a calorie-restricted diet regime by a healthcare provider, and/or (iii) is treated for stroke recovery by being administered the activator and without being on a calorie-restricted diet regime.

In embodiments, the activator of Integrated Response Pathway is administered at least daily for a period of at least a week subsequent to the subject having a stroke.

In embodiments, the activator of Integrated Response Pathway is administered at least daily for a period of at least four weeks subsequent to the subject having a stroke.

In embodiments, the activator of Integrated Response Pathway is 2-deoxyglucose and is administered to a human subject at an amount of at least 50 μg/Kg body weight/day.

In embodiments, the activator of Integrated Response Pathway is 2-deoxyglucose and is administered to a human subject at an amount of at least 100 μg/Kg body weight/day.

In embodiments, the activator of Integrated Response Pathway is 2-deoxyglucose and is administered to a human subject at an amount of at least 500 μg/Kg body weight/day.

In embodiments, the activator of Integrated Response Pathway is 2-deoxyglucose and is administered to a human subject at an amount of at least 1 mg/Kg body weight/day.

In embodiments, the administration of the treatment does not elicit hypoglycemia in the subject. In embodiments, the administration does not elicit hepatic effects.

In embodiments, the administration effects an improvement in stroke-associated memory defect in the subject.

In embodiments, the administration effects an improvement in stroke-associated learning defect in the subject.

In embodiments, the administration effects an improvement in stroke-associated spatial long-term memory defect in the subject.

In embodiments, the administration effects an improvement in stroke-associated sensory defect in the subject.

In embodiments, the administration effects an improvement in stroke-associated motor function defect in the subject.

An improvement is positive or ameliorative change in one or more quantitative parameters, or qualitative parameters, e.g., as determined by a healthcare provider, relative to an untreated equivalent.

In embodiments, the method treats hemorrhagic stroke in the subject.

In embodiments, the method treats ischemic stroke in the subject.

In embodiments, the administration of 2-deoxyglucose directly into the CNS treats Alzheimer's in the subject.

A method of treating a neurodegenerative disease in a subject comprising administering to the subject an amount of an activator of 2-deoxyglucose effective to treat a neurodegenerative disease.

In embodiments, the neurodegenerative disease is stroke, Parkinson's disease, Alzheimer's disease, epilepsy, or Huntington's disease.

In embodiments, the methods comprise administering the activator parenterally or systemically.

In embodiments, the methods comprise administering the activator orally or intravenously.

In embodiments, the methods comprise administering the activator directly into the CNS of the subject. In embodiments, the methods comprise administering the activator intranasally the subject. In embodiments, the methods comprise administering the activator via the upper nasal epithelium of the subject. In embodiments, administering the activator directly into the CNS of the subject does not result in systemic hypoglycemia, and/or reduces systemic effects as compared to administration to the subject external to the CNS. In embodiments, the activator is administered directly into a CSF pathway. In embodiments, the activator is injected directly into a CSF pathway. In embodiments, the activator is administered directly into the brain. In embodiments, the activator is injected directly the brain.

A pharmaceutical composition comprising a pharmaceutically acceptable carrier and an activator of an Integrated Stress Response pathway effective to increase phosphorylation of an Eukaryotic Initiation Factor 2 alpha (eif2alpha) for reducing ferroptosis.

A pharmaceutical composition comprising a pharmaceutically acceptable carrier and an activator of an Integrated Stress Response pathway effective to increase phosphorylation of an Eukaryotic Initiation Factor 2 alpha (eif2alpha) for enhancing gene expression associated with learning, memory and/or plasticity in the human CNS.

In embodiments, the activator is 2-deoxyglucose.

A pharmaceutical composition comprising a pharmaceutically acceptable carrier and an inhibitor of N-linked glycosylation for promoting plasticity and/or adaptation to injury in the human CNS.

In embodiments, the inhibitor is tunicamycin.

In embodiments, the pharmaceutical composition ameliorates ferroptosis.

A method of treating a neurodegenerative disease in a subject comprising administering to the subject an amount of an inhibitor of N-linked glycosylation thereby treating the neurodegenerative disease. In embodiments, the N-linked glycosylation inhibited is in an endoplasmic reticulum. In embodiments, the N-linked glycosylation inhibited is in cytosol. In embodiments, the methods comprise administering the inhibitor directly into the CNS of the subject. In embodiments, the methods comprise administering the inhibitor intranasally the subject. In embodiments, the methods comprise administering the inhibitor via the upper nasal epithelium of the subject. In embodiments, administering the inhibitor directly into the CNS of the subject does not result in systemic hypoglycemia, and/or reduces systemic effects as compared to administration to the subject external to the CNS. In embodiments, the inhibitor is administered directly into a CSF pathway. In embodiments, the inhibitor is injected directly into a CSF pathway. In embodiments, the inhibitor is administered directly into the brain. In embodiments, the inhibitor is injected directly the brain.

In embodiments, the inhibitor reduces ferroptosis.

In embodiments, the inhibitor is tunicamycin, 1-Deoxynojirimycin, or an indolizine.

In embodiments, the inhibitor is a small molecule.

A method of treating a neurodegenerative disease in a subject is provided comprising administering to the subject an amount of an inhibitor of N-linked glycosylation thereby treating the neurodegenerative disease. In embodiments, the N-linked glycosylation inhibited is in an endoplasmic reticulum. In embodiments, the N-linked glycosylation inhibited is in cytosol. In embodiments, the methods comprise administering the inhibitor directly into the CNS of the subject. In embodiments, the methods comprise administering the inhibitor intranasally the subject. In embodiments, the methods comprise administering the inhibitor via the upper nasal epithelium of the subject. In embodiments, administering the inhibitor directly into the CNS of the subject does not result in systemic hypoglycemia, and/or reduces systemic effects as compared to administration to the subject external to the CNS. In embodiments, the inhibitor is administered directly into a CSF pathway. In embodiments, the inhibitor is injected directly into a CSF pathway. In embodiments, the inhibitor is administered directly into the brain. In embodiments, the inhibitor is injected directly the brain.

In embodiments, the inhibitor inhibits a flippase, oligosaccharyltransferase (OST), or a glucosidase.

In embodiments, the glucosidase is a glucosidase II.

In embodiments, the inhibitor of glucosidase II is an siRNA, miglitol, N-butyl-deoxynojirimycin (miglustat), N-nonyldeoxynojirimycin (NN-DNJ), celgosivir, and acarbose.

In embodiments, the inhibitor is tunicamycin, 1-Deoxynojirimycin, or an indolizine.

In embodiments, the inhibitor is an inhibitor of OST.

In embodiments, the inhibitor is an aminobenzamide-sulfonamide NGI-1.

In embodiments, the inhibitor is a peptidyl inhibitor.

In embodiments, the inhibitor is a peptidyl inhibitor comprising the sequence NXS/T.

In embodiments, the administration of the treatment does not elicit hypoglycemia in the subject.

In embodiments, the method treats hemorrhagic stroke in the subject. In embodiments, the method treats ischemic stroke in the subject.

In embodiments, the neurodegenerative disease is stroke, Parkinson's disease, Alzheimer's disease, epilepsy, or Huntington's disease.

In embodiments, the methods comprise administering the inhibitor directly into the CNS of the subject. In embodiments, the methods do not comprise administering the inhibitor into the subject externally to the subject's CNS.

In embodiments, the inhibitor of N-linked glycosylation is a peptidyl inhibitor. Wherein the inhibitor is a peptide of 10 amino acids or less comprising the sequence NXS/T (where X is any amino acid, S is serine and T is threonine). Wherein the inhibitor is a peptide of 10 amino acids or less comprising the sequence NXS/T (where X is any amino acid, S is serine and T is threonine) linked to a TAT domain. In an embodiment, the peptide of 10 amino acids or less comprising the sequence NXS/T has the same sequence as a 10 amino acid or less portion of a human protein or polypeptide. In an embodiment, X is F, G, I, S, T or V. In embodiments, the peptide of 10 amino acids or less comprising the sequence NXS/T is 10, 9, 8, 7, 6, 5, 4 or 3 amino acids in length. In an embodiment, the TAT domain is, or comprises, YGRKKRRQRRR (SEQ ID NO: 23).

In an embodiment, the peptidyl inhibitor comprises a first portion which is a peptide of 10 amino acids or less comprising the sequence NXS/T (where X is any amino acid, S is serine and T is threonine) linked to a second portion which is a TAT domain, and optionally a third portion which is a targeting sequence selected from the group consisting of NA1-Tat NR2B9c, PTP-Sigma, GluR2-Gapdh inhibitor sequence, SDK-5 inhibitor sequence, and SS31 sequence. In an embodiment, the first, second and third portion are joined as a single peptide.

In embodiments, the inhibitor of N-linked glycosylation is a plant alkaloid. Examples include castanospermine (from the seed of the Australian chestnut tree, Castanosperum australe), which inhibits α-glucosidases I and II, australine (also from C. australe), which preferentially inhibits α-glucosidase I, and deoxynojirimycin (from Streptomyces species).

In embodiments, the inhibitor is a small molecule.

Flippases can be inhibited by siRNA and RNAi mechanisms, as well as antibodies, flippase-binding antibody fragments and nanobodies. Oligosaccharyltransferase can be inhibited by siRNA and RNAi mechanisms, as well as antibodies, OST-binding antibody fragments and small molecules such as the aminobenzamide-sulfonamide NGI-1 that targets the OST complex (see, e.g., Puschnik, A. S. et al. A small-molecule oligosaccharyltransferase inhibitor with pan-flaviviral activity. Cell Rep. 21, 3032-3039 (2017), hereby incorporated by reference). OST can also be inhibited by peptidyl inhibitors, such as cyclo(hex-Amb-Cys)-Thr-Val-Thr-Nph-NH₂. Inhibitors of glucosidase II include miglitol, N-butyl-deoxynojirimycin (miglustat), N-nonyldeoxynojirimycin (NN-DNJ), celgosivir, and acarbose.

A method of treating a subject for a cancer comprising administering to the subject an amount of an activator of an Integrated Stress Response pathway effective to increase phosphorylation of an eif2alpha and thereby reduce ferroptosis in the subject so as to treat the cancer.

In embodiments of the methods, the subject is a human.

In embodiments, the eif2alpha is human eif2alpha.

In embodiments, the Integrated Stress Response is a human Integrated Stress Response.

A method of protecting a cell in a plant from heat stress comprising treating the plant with an amount of an activator of an Integrated Stress Response pathway effective to increase phosphorylation of an eif2alpha and thereby reduce ferroptosis in the plant and protect from heat stress.

As used herein, a predetermined control amount is a value decided or obtained, usually beforehand, as a control. The concept of a control is well-established in the field, and can be determined, in a non-limiting example, empirically from non-afflicted subjects (versus afflicted subjects, including afflicted subjects having different grades of the relevant affliction), and may be normalized as desired (in non-limiting examples, for volume, mass, age, location, gender) to negate the effect of one or more variables.

“And/or” as used herein, for example with option A and/or option B, encompasses the separate embodiments of (i) option A, (ii) option B, and (iii) option A plus option B.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### Experimental Details

We focused on addressing the unanswered questions and unknowns discussed in the Background by utilizing strategies based on multiple in-vitro and in-vivo models. We assessed how plasticity genes known to play important roles in learning and memory respond to 2-DG treatment. We also investigated the consequences of 2-DG on learning and memory in 5×FAD mice and on sensory-motor function in mice models of ischemic and hemorrhagic stroke. Since protein synthesis plays important role in learning and memory, we next investigated if the beneficial effects of 2-DG are transcription dependent or dependent on some post-transcription process. Furthermore, we identified a key mediating player involved in translating the metabolic effects of 2-DG into enhanced plasticity program that plays important role in 2-DG led improvement in learning and memory in AD and functional recovery in stroke.

### 2-Deoxyglucose Induces the Plasticity Program Involved in Learning and Memory and Repair

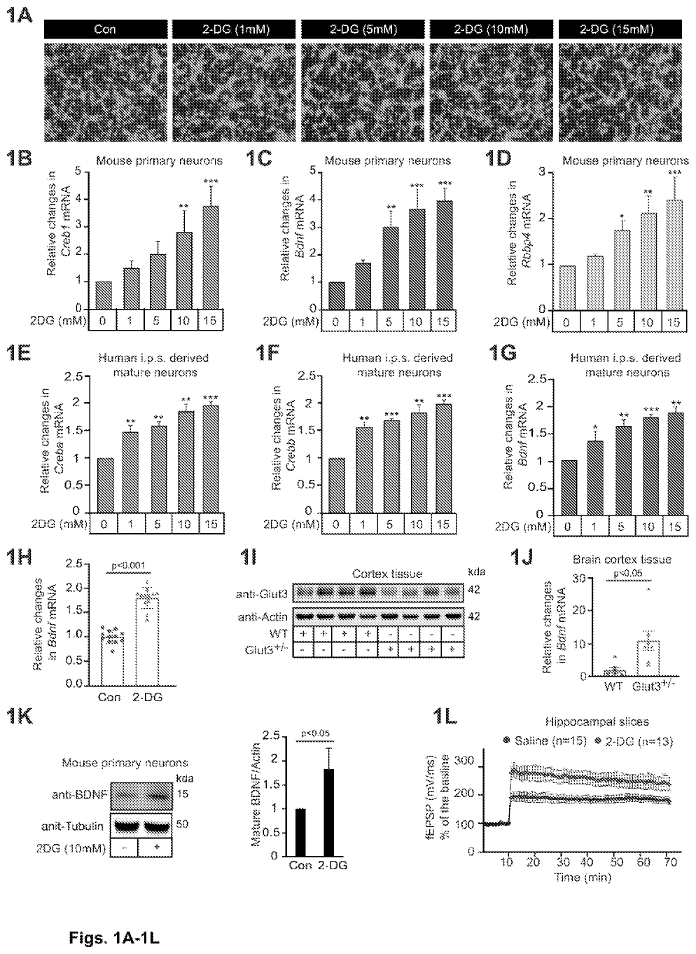

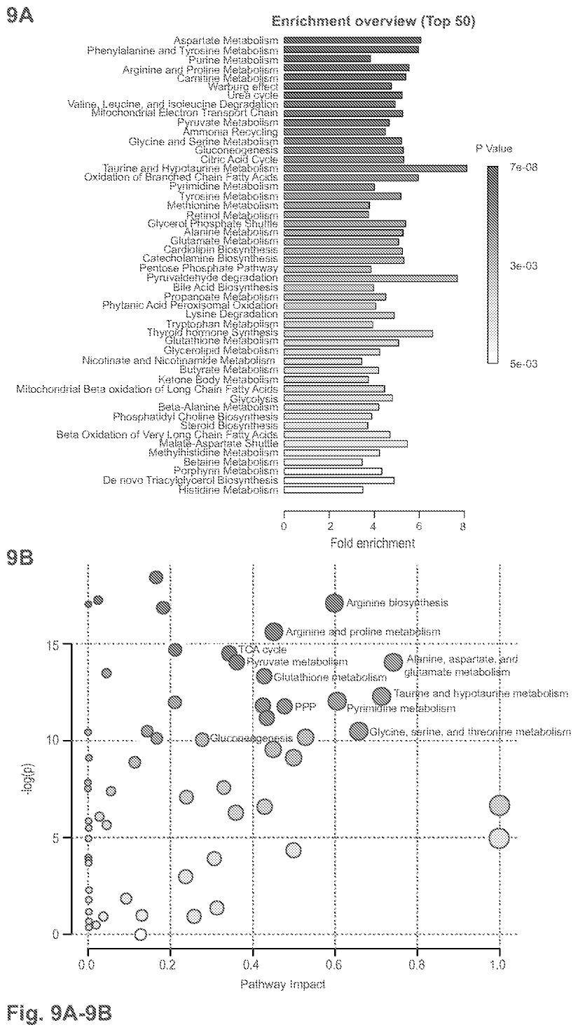

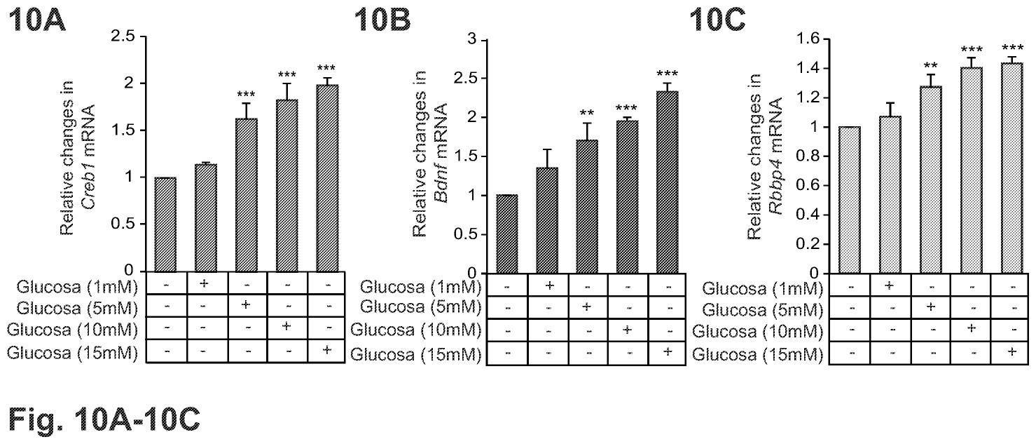

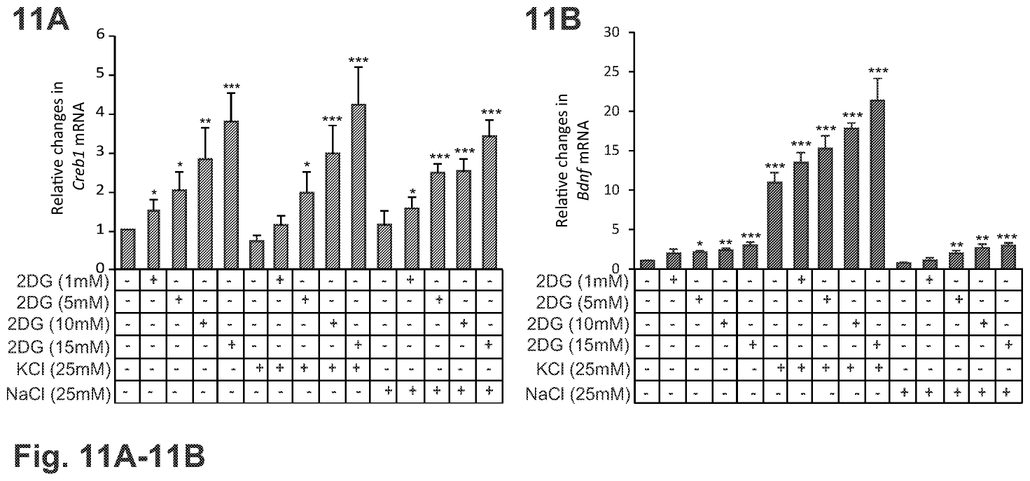

2-DG has been previously hypothesized to mimic the beneficial effects of intermittent fasting and calorie restriction at systems level by inducing mild energetic stress (Wan et al., 2004). Moreover, the dietary supplementation of 2-DG in rats has been shown to mimic the physiological effects of calorie restriction such as decrease in insulin level, body temperature, blood pressure, heart rate, plasma glucose level, and enhanced cardiovascular and neuroendocrine adaptation to stress (Lane et al., 1998; Wan et al., 2004). At cellular level, 2-DG decreases the utilization of glucose, the main substrate for the energy production by inhibiting the glycolytic pathway. Considering glucose as a dominant energy source for neuronal function and viability, we first assessed if 2-DG treatment in cultured mouse primary neurons for 24 h increases neuronal cell death or not. The live-dead assay showed no toxicity with all tested concentrations of 2-DG between 1 mM to 15 mM (FIG. 1A). It was likely that 2-DG induced a compensatory increase in the utilization of alternate energy substrates such as glutamate and other amino acids present in the culture medium to maintain the optimal functioning and viability of neurons (FIGS. 9A and 9B). Next, we tested the response of neurons to 2-DG with respect to the expression of plasticity genes such as Creb1, Bdnf, and Rbbp4, which are considered to play critical roles in learning and memory. We began with three testable unbiased hypotheses. First, considering glucose as a key energy substrate, we investigated if 2-DG might decrease the plasticity gene expression. Second, with the availability of the alternate energy substrates, the expression of plasticity genes might not be affected in response to increasing doses of 2-DG treatments. Third, neurons might respond by inducing a compensatory adaptive transcriptional program as an attempt to re-establish homeostasis and thereby, 2-DG might increase the plasticity gene expression. Interestingly, we found that 2-DG treatment resulted in a dose-dependent increase in the expression of plasticity genes such as Creb1, Bdnf, and Rbbp4 (FIGS. 1B-1D). We further confirmed the compensatory increase in plasticity gene program in response to diminished glucose utilization by using another glycolytic inhibitor, glucosamine. Notably, increasing concentrations of glucosamine also led to a dose dependent increase in the expression of plasticity genes such as Creb1, Bdnf, and Rbbp4 (FIGS. 10A-10C). We next tested the same hypotheses in depolarized neurons as learning and memory requires neuronal depolarization. The genetic plasticity response of neurons was still preserved (FIGS. 11A and 11B). Next, we confirmed these hypotheses in human i.p.s. derived mature cortical neurons. Notably, the gene plasticity response of neurons was still the same (FIGS. 1E-1G). Thus, both mouse and human in-vitro data indicated that 2-DG treatment enhances plasticity gene program.

Next, we focused mainly on Bdnf as this is a well-established neurotrophic growth factor which plays important role in memory. Using Bdnf as a surrogate marker of plasticity gene program, we tested if acute treatment of 2-DG (Intraperitoneal injection for 6 h) can induce the expression of plasticity gene program in the brain cortex or not. Interestingly, 2-DG led to a significant increase in Bdnf gene expression mouse brain cortex indicating that 2-DG induces the adaptive plasticity program not just in isolated neurons but also in the in-vivo conditions (FIG. 1H). Since 2-DG is known to work by inhibiting the glucose uptake and utilization at-least at the cellular level, we next compared Bdnf gene expression in the brain cortex of wild type and Glut3 heterozygous knockout mice (Glut3^{+/-}) that are partially defective in neuronal glucose transporter, Glut3. Importantly, Bdnf gene expression in the brain cortex of Glut3 heterozygous knock out mice was significantly higher as compared to wild type mice indicating that chronic decrease in neuronal glucose uptake leads to upregulation of compensatory adaptive plasticity gene program (FIGS. 1I and 1J). These Glut3^{+/-} mice show completely normal cognitive function (Stuart et al., 2011) most likely due to upregulated Bdnf expression. The Glut3 homozygotes are embryonically lethal indicating the dominant effect of lack of neuronal glucose uptake in these mice. This finding further supported that increase in Bdnf gene expression seen in the mouse brain cortex in response to acute treatment of 2-DG (Intraperitoneal injection for 6 h) could likely be due to decrease in neuronal glucose uptake.

Since the contribution of this growth factor towards long term potentiation and learning and memory has been attributed to the mature Bdnf protein, we next assessed the effect of 2-DG treatment on the mature BDNF protein in mouse primary neurons. Interestingly, 2-DG led to increase in mature BDNF protein as well (FIG. 1K). Since the long-term potentiation (LTP) provides a neuro-physiological connection between the plasticity gene program (A simple biological process) and the learning and memory (A complex biological process), we next tested the effect of 2-DG on the LTP in submerged hippocampal slices. We found a significant increase in LTP in submerged hippocampal slices in response to acute treatment of 2-DG (10 mM) for 6 h (FIG. 1L). This finding affirmed the in-vitro finding of 2-DG led increase in expression of Bdnf and other plasticity genes known to be involved in learning and memory, repair, and cell survival program.

### 2-DG Normalizes Defect in Learning and Memory in 5×FAD Mice

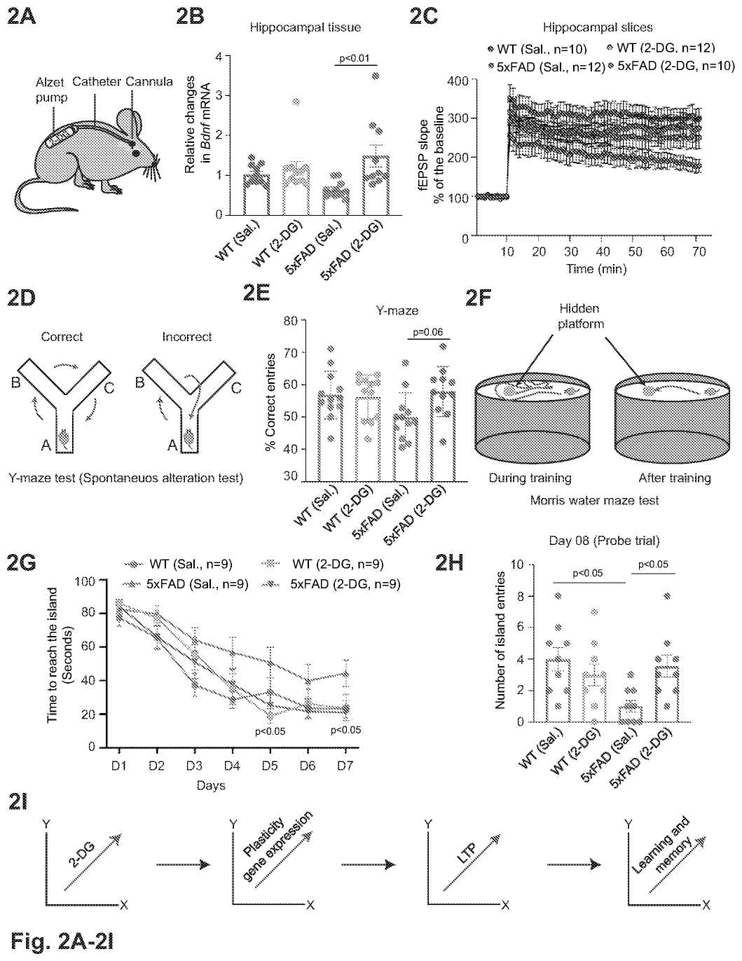

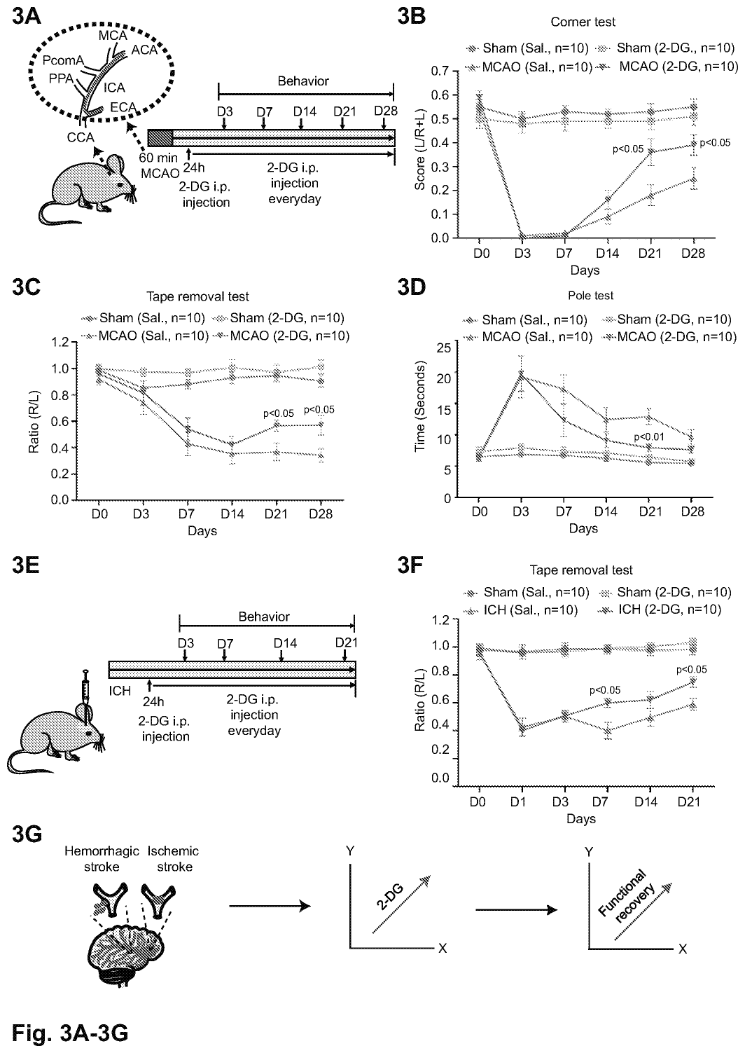

Since calorie restriction regimes have been found to improve learning and memory in many preclinical (Gudden et al., 2021; Halagappa et al., 2007; Liu et al., 2019) and clinical studies (Horie et al., 2016; Leclerc et al., 2020; Ooi et al., 2020; Ota et al., 2019; Smith et al., 2010; Witte et al., 2009) and our in-vitro, ex-vivo as well as in-vivo models showed 2-DG led significant increase in plasticity gene program known to play important role in learning and memory, we, next, examined the effects of 2-DG directly on learning and memory. We first aimed to allow direct release of 2-DG in the ventricles of the brain and assess its effect on the improvement of learning and memory deficit observed in six months old 5×FAD mouse, a well-established model of AD, which expresses human APP and PSEN1 transgenes with five AD-linked mutations. 5×FAD mouse develops amyloid pathology very quickly. This widely-used mouse shows decline in spatial memory in Y-maze (Spontaneous alternation test) and Morris water maze at around 6 months of age and decrease in long term potentiation a little before. We implanted Alzet (osmotic) pump filled with 2-DG below the neck of mice and connected its catheter tube to the brain through intra-cerebro-ventriclular (ICV) injection (FIG. 2A). This allowed a slow release of 2-DG in the brain for four weeks. Alzet pump filled with saline worked as treatment control. After four weeks of slow release of 2-DG into the ventricles of the brain, we assessed changes in short-term and long-term spatial memory using Y-maze task and Morris water maze task and, thereafter, euthanized mice and dissected out the brain hippocampi and used few hippocampal slices for LTP study and other slices for Bdnf gene expression study.

Notably, 2-DG treatment normalized Bdnf gene expression as well as LTP in 5×FAD mice to the level almost similar as wild type mice treated with saline (FIGS. 2B—2C). Interestingly, 2-DG didn't lead to nootropic upregulation of Bdnf gene expression and LTP in wild type mice most likely because of the re-establishment of homeostasis (FIGS. 2B and 2C). When we tested changes in spatial short-term memory through spontaneous alternation test in Y-maze and spatial long-term memory in Morris water maze, we again found that 2-DG normalized these memories in 5×FAD mice to the level almost similar as wild type mice treated with saline. Again, 2-DG did not show any nootropic increase in wild type mice in both Y-maze task and Morris water maze task (FIGS. 2D-2H). These data suggested that the calorie restriction mimetic, 2-DG not only upregulates plasticity gene expression and long-term potentiation but also normalizes the learning and memory defect seen in AD (FIG. 21).

2-DG Treatment after Stroke Improves Sensory and Motor Function

Calorie restriction has been shown to improve functional outcome in many pre-clinical models of acute neurological disorders such as stroke and traumatic brain injury (TBI) (Roberge et al., 2008; Rubovitch et al., 2019). Based on the findings that calorie restriction mimetic, 2-DG upregulated the plasticity gene program that plays important role not only in learning and memory but also in repair and cell survival programs required for functional recovery after acute injuries such as stroke and TBI, we hypothesized that 2-DG will improve functional recovery if given in recovery phase after stroke. We found the beneficial effects of direct release of 2-DG in the brain with respect to learning and memory, but this could pose a practical limitation with respect to using 2-DG as a therapeutic in the clinic. Therefore, we tested intraperitoneal route of 2-DG injection and hypothesized that it should also provide the benefit as it can easily cross the blood brain barrier and reach the brain. We injected 2-DG (10 mg/Kg) intraperitoneally at 24 h after inducing transient ischemic stroke through middle cerebral artery occlusion (MCAO) in the mouse brain (FIG. 3A) and everyday thereafter for 4 weeks and assessed the sensory and motor functions using Corner turn test, tape removal test, and pole test at different intervals. We found that 2-DG treatment after ischemic stroke led to significant improvement in corner test, tape removal test, and pole test in later phase of the recovery (FIGS. 3B-3D). We further confirmed 2-DG led improvement in functional recovery in hemorrhagic stroke as well. We injected 2-DG (10 mg/Kg) intraperitoneally at 24 h after inducing hemorrhagic stroke using collagenase (An enzyme that induces hemorrhage through disruption of the basal lamina of the blood vessels) in the mouse brain (FIG. 3E) and everyday thereafter for 3 weeks and assessed the sensory and motor functions using tape removal test at different intervals. 2-DG treatment after hemorrhagic stroke also led to significant improvement from day 7 onwards (FIG. 3F). These findings suggested that 2-DG enhances functional recovery after stroke most likely by engaging the plasticity/repair program (FIG. 3G).

### Energy Sensing is not Necessary for 2-DG LED Induction of Plasticity Program

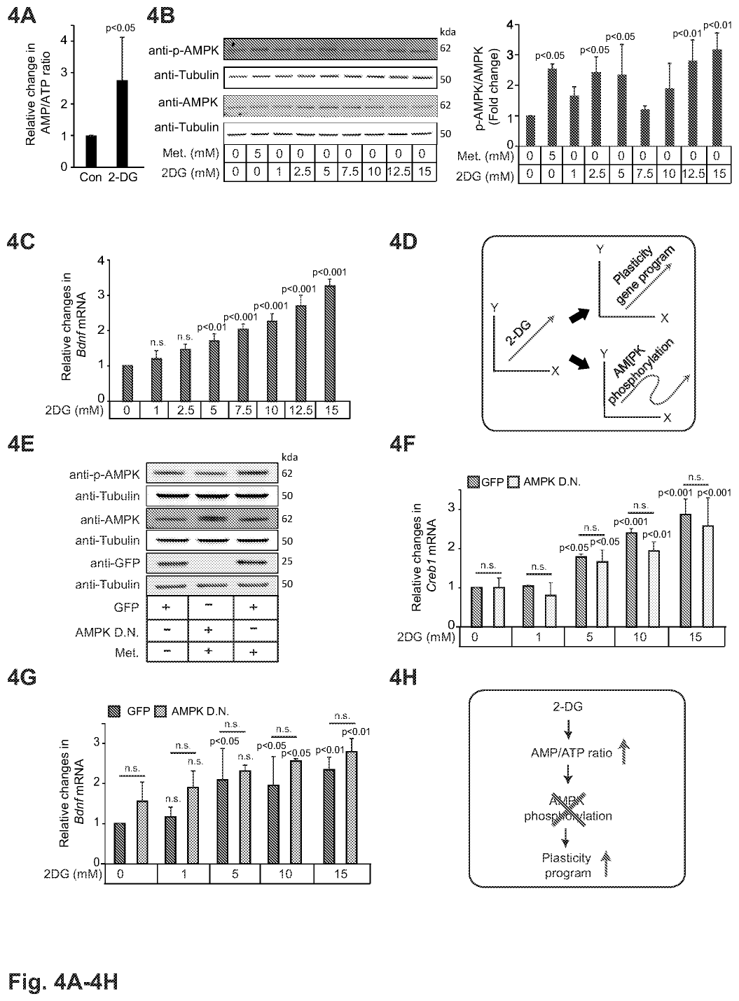

Glucose is considered the most dominant source of energy for neurons. Since 2-DG inhibits the utilization of glucose by inhibiting the glycolysis, it does affect the production of ATP as indicated by 2-DG led increase in AMP-ATP ratio (FIG. 4A). AMPK is considered a very sensitive fuel gauze as it senses the change in AMP/ATP ratio and signals the cellular machinery of this change by increasing its own phosphorylation at threonine 172. We asked if energy sensing or AMPK phosphorylation is the key molecular mechanism mediating the crosstalk between 2-DG led changes in energy status with homeostatic changes in plasticity genes. We hypothesized that if this is the case, there should be a strong correlation between 2-DG led changes in AMPK phosphorylation and plasticity gene expression. When we treated primary cortical neurons with increasing concentrations of 2-DG (1-15 mM), changes in the level of AMPK phosphorylation at threonine 172 was biphasic (FIG. 4B) but changes in Bdnf gene expression was does dependent (FIG. 4C). This lack of correlation between AMPK phosphorylation and plasticity gene expression suggested towards the possibility that 2-DG led changes in plasticity gene expression might not be related to changes in energy sensing (FIG. 4D). Next, to confirm if AMPK phosphorylation mediates this adaptive process or not, we knocked down AMPK phosphorylation using adenoviral expression of dominant negative AMPK (FIG. 4E). Metformin was used to induce AMPK phosphorylation as a positive control. Interestingly, knocking down AMPK phosphorylation did not block 2-DG led increase in Creb1 and Bdnf gene expression (FIGS. 4F and 4G). This finding confirmed that AMPK phosphorylation or energy sensing does not mediate 2-DG led increase in plasticity genes program (FIG. 4H).

### 2-DG LED Induction of Plasticity Program is Transcription Dependent

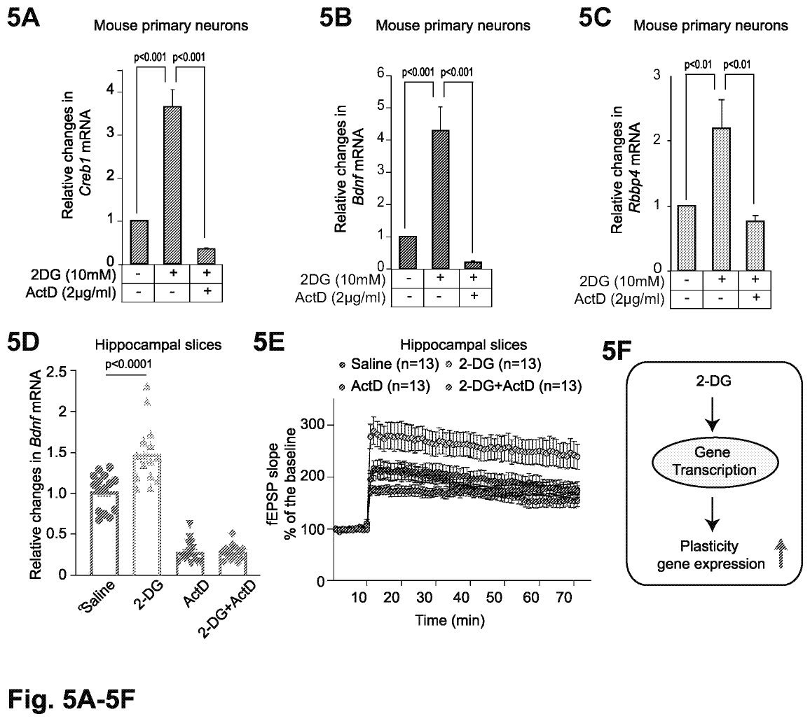

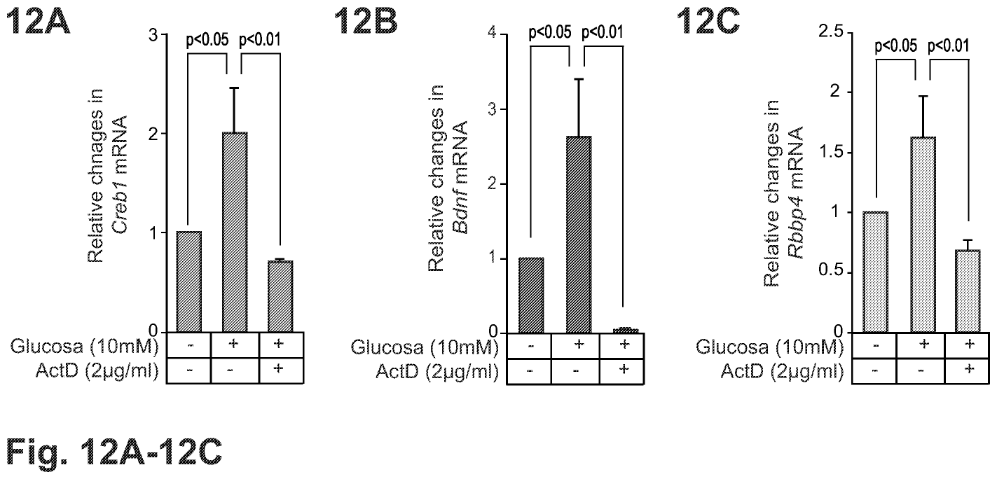

Since glucose is not just an energy substrate but also plays important role in a number of transcriptional and post-transcriptional biological processes (Kumar, 2019), we asked if 2-DG led increase in plasticity gene program and LTP is transcription dependent or transcription independent. To this end, we co-treated primary neurons with 2-DG and transcriptional inhibitor, actinomycin D (ActD) and assessed changes in the expression of plasticity genes. We found that inhibiting transcription with ActD completely blocked 2-DG led increase in plasticity genes such as Creb1, Bdnf, and Rbbp4 (FIGS. 5A-5C). Similarly, glucosamine induced expression of plasticity genes were also transcription dependent (FIGS. 12A-12C). Next, we tested if ActD could block the 2-DG led increase in LTP. First, we confirmed that the ActD (1 μg/ml) was able to block 2-DG led increase in Bdnf gene expression in the hippocampal slices (FIG. 5D). Then, we checked if the same concentration of ActD was able to block the LTP or not. Interestingly, we found that ActD completely blocked the 2-DG led induction in LTP (FIG. 5E). These findings suggested that 2-DG led induction of plasticity gene program and LTP is transcription dependent (FIG. 5G).

### High Throughput RNA Sequencing Shows Endoplasmic Reticulum as the Target Organelle

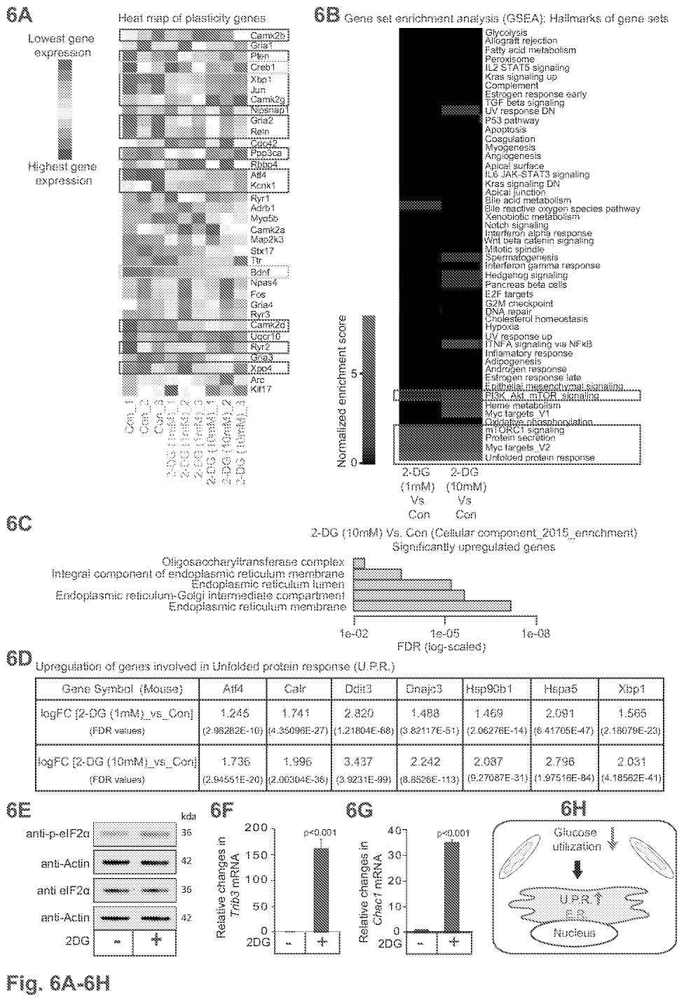

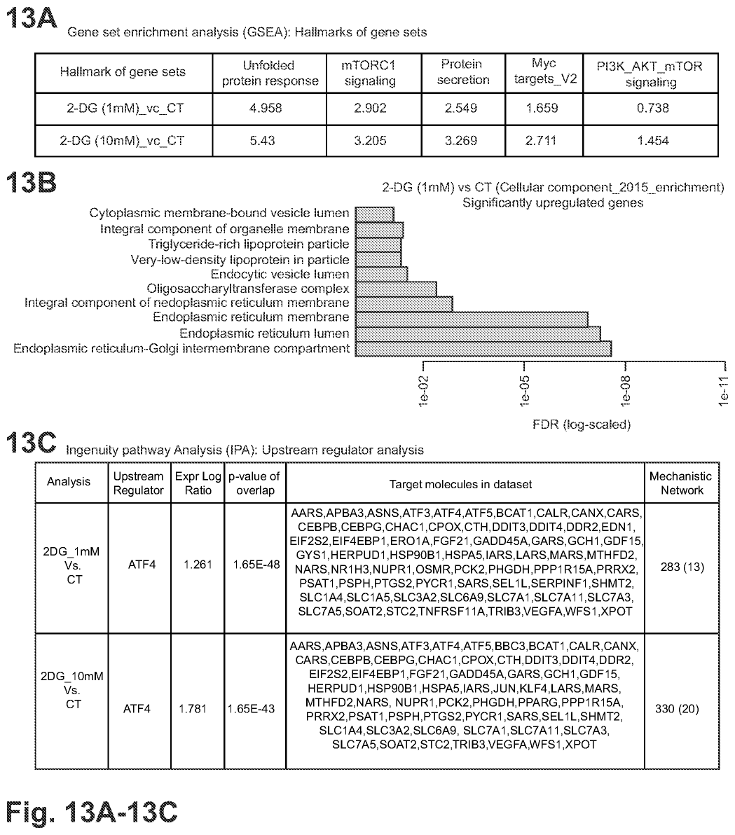

Since glucose regulates several transcription dependent processes, instead of following a candidate approach, we undertook the unbiased high throughput RNA Sequencing approach with two main aims: 1) to see if 2-DG treatment leads to changes in a broad plasticity gene program or just few candidate plasticity genes and 2) to find most dominant gene signature in response to 2-DG treatment to identify biological process most sensitive to 2-DG treatment. We performed an unbiased RNA sequencing of primary neurons treated with either 1 mM or 10 mM 2-DG for 6 h. We found an increase in the expression of not just Creb1 and Bdnf genes but an upregulation of a broad plasticity gene program (FIG. 6A). Importantly, gene set enrichment analysis (GSEA) showed maximal upregulation of the gene signature related to unfolded protein response (UPR) in response to 2-DG treatment (FIG. 6B and FIG. 13A). Moreover, cellular component enrichment analysis showed a significant enrichment of genes related to endoplasmic reticulum (FIG. 6C and FIG. 13B) indicating endoplasmic reticulum as the key affected organelle in response to 2-DG treatment. Of note, all genes related to the unfolded protein response showed dose dependent increase in response to 1 mM and 10 mM concentrations of 2-DG (FIG. 6D). Endoplasmic reticulum (ER) is a key organelle involved in protein homeostasis by ensuring proper folding of proteins with the help of chaperones. Glucose along with mannose play important role within endoplasmic reticulum by contributing to proper folding of proteins through N-linked glycosylation. A defect in proper folding of proteins leads to a buildup of unfolded/misfolded proteins, which is called unfolded protein response. This is an adaptive response, which triggers several downstream signaling cascades leading to restoration of protein homeostasis. One of the downstream responses includes the phosphorylation of eIF2α through PERK, which slows down the synthesis of new proteins to reduce the burden on ER by interfering with translation. Through immunoblotting, we confirmed that the dominant UPR gene signatures seen in the high throughput RNA sequencing was able to induce the downstream signaling cascade by inducing eIF2α phosphorylation (FIG. 6E). Interestingly, eIF2α phosphorylation on one hand decreases general translation but, on other hand, induces the paradoxical translation of ATF4. ATF4 is a transcription factor, which regulates the expression of a cassette of genes involved in plasticity, long term potentiation and learning and memory (Pasini et al., 2015). Ingenuity pathway analysis for upstream regulators showed upregulation of around 200-300 ATF4 target genes in response to 2-DG treatment (FIG. 13C). We confirmed the activation of ATF4 by assessing the expression of two ATF4 target genes, Trib3 and Chac1 (FIGS. 6F and 6G). These data suggested that ER is the key target organelle and UPR as the key biological process affected by 2-DG treatment (FIG. 6H).

### ATF4 Mediates 2-DG LED Increase in the Expression of Bdnf and Likely Other Plasticity Genes

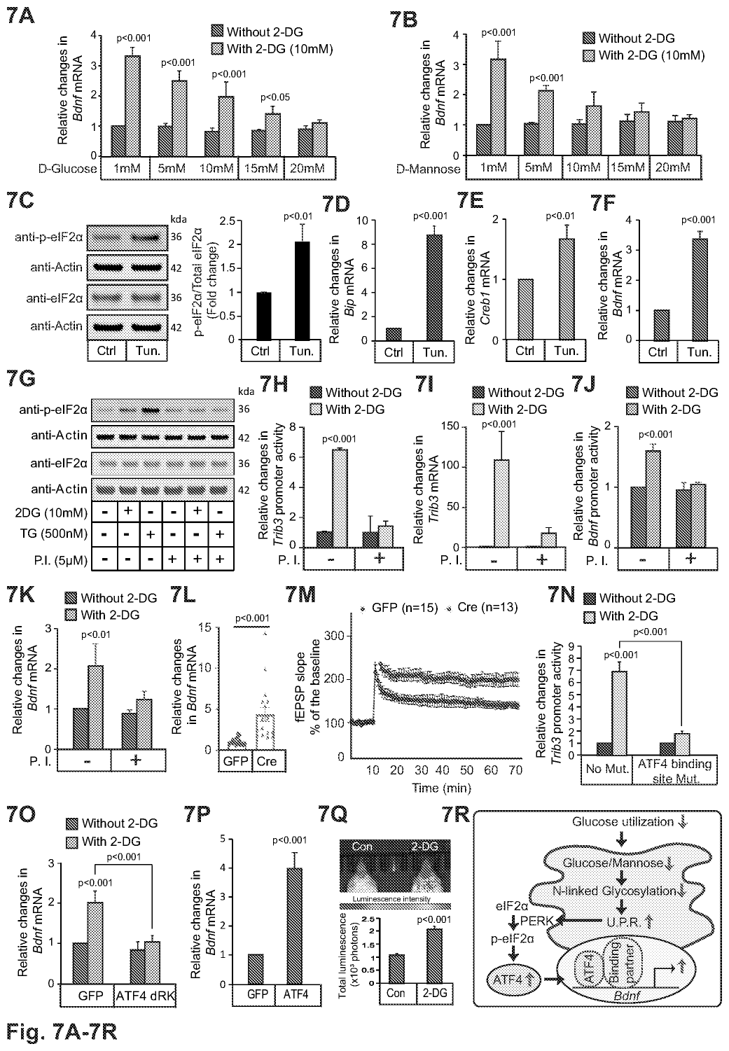

The high throughput RNA sequencing data indicated UPR as the main biological process affected by 2-DG treatment, but the question remained if unfolded protein response mediates the glucose sensing of plasticity genes. 2-DG treatment could affect unfolded protein response by depleting D-glucose or D-mannose that support proper folding of proteins through N-linked glycosylation. We hypothesized if a decrease in N-linked glycosylation is necessary for 2-DG led induction in plasticity genes, for instance, Bdnf gene expression, increasing either D-glucose or D-mannose in the normal media covering primary neurons will decrease the 2-DG led induction of Bdnf gene expression. Interestingly, we found that increasing concentrations of D-glucose or D-mannose decreased 2-DG led increase in Bdnf gene expression in a dose dependent manner indicating that the N-linked glycosylation affected by changes in the availability of these biomolecules is a necessary trigger for 2-DG led increase in Bdnf gene expression (FIGS. 7A and 7B). Next, we examined if N-linked glycosylation is sufficient in the upregulation of plasticity genes or not by treating primary neurons with tunicamycin (3 μM), a well-established pharmacological inhibitor of N-linked glycosylation (Surani, 1979), for 6 h. An increase in phosphorylation of eIF2α (FIG. 7C) and an increase in gene expression of chaperone Bip (FIG. 7D) confirmed the tunicamycin led inhibition of N-linked glycosylation and consequent upregulation of UPR. Interestingly, tunicamycin treatment led to an increase in the expression of plasticity genes, Creb1 and Bdnf (FIGS. 7E and 7F) indicating that inhibition of N-linked glycosylation is sufficient in inducing plasticity gene program.

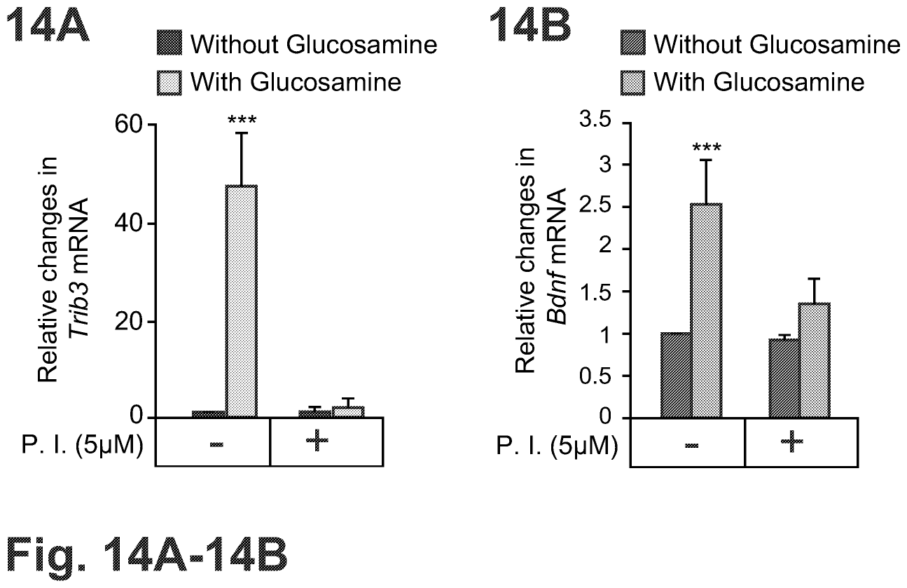

Next, we tested if PERK-eIF2α-ATF4 axis downstream of UPR activation is necessary for 2-DG led upregulation of Bdnf gene expression or not. To test this, we treated primary neurons with GSK2606414, a selective PERK inhibitor (Axten et al., 2012) that blocks PERK led eIF2α phosphorylation. The concentration of GSK2606414 that completely blocked 2-DG led increase in PERK mediated cIF2a phosphorylation (FIG. 7G) and promoter activity and expression of downstream ATF4 target gene, Trib3 in primary neurons (FIGS. 7H and 71) also blocked 2-DG led increase in the promoter activity and expression of Bdnf gene (FIGS. 7J and 7K). Similarly, glucosamine induced expression in Trib3 and Bdnf genes were blocked by the specific PERK inhibitor, GSK2606414 (FIGS. 14A and 14B). This showed that 2-DG led decrease in glucose uptake and utilization leads to upregulation in the expression of Bdnf and likely other plasticity genes through PERK-eIF2α-ATF4 axis as a necessary mediator.

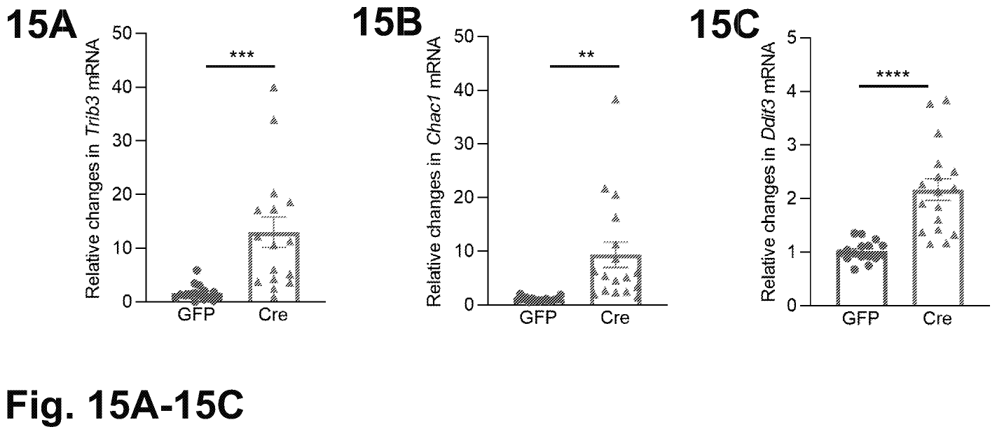

We, next, aimed to confirm if eIF2α phosphorylation is sufficient to upregulate plasticity genes and LTP or not. The protein phosphatase 1 (pp1) regulatory subunit 15 (Ppp1R15) specifically catalyzes the dephosphorylation of eIF2a. There are two isoforms of pp1 in mammals categorized on the basis of the regulatory subunit coupled to it: the stress inducible Ppp1R15a and the constitutive Ppp1R15b (So et al., 2015). The knockdown of Ppp1R15b has been shown to induce constitutive activation of eIF2α phosphorylation (So et al., 2015). We transiently knocked down Ppp1r15b in the hippocampus of Ppp1r15b mouse, a homozygous floxed mouse by intracranial injection of AAV8-Cre and verified the constitutive activation of eIF2α phosphorylation by activation of downstream ATF4 target genes such as Trib3, Chac1, and Ddit3 in the hippocampus (FIGS. 15A-15C). Notably, constitutive activation of eIF2a phosphorylation led to a significant increase of Bdnf gene expression in the hippocampus (FIG. 7L) along with an increase in LTP in these hippocampal slices (FIG. 7M). These findings suggested that eIF2α phosphorylation is sufficient in inducing plasticity gene program and LTP.

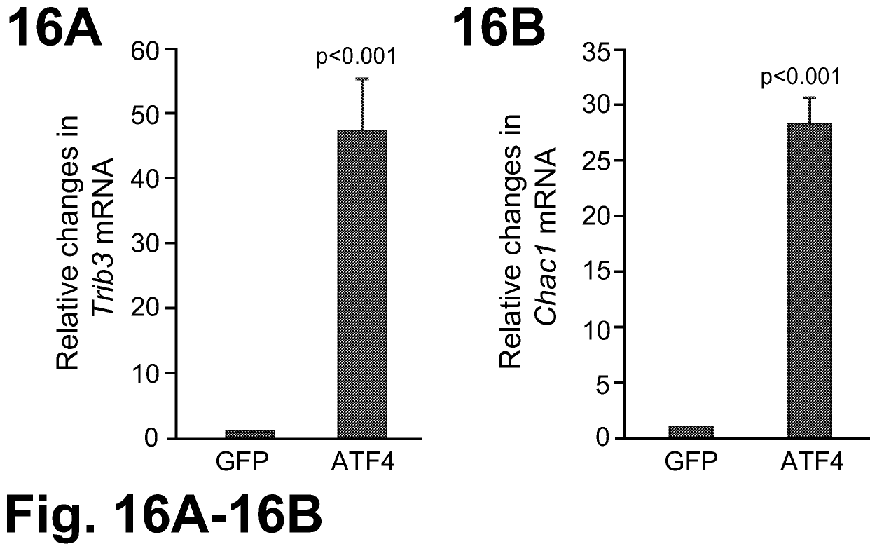

eIF2α phosphorylation, on one hand, represses general translation likely to reduce protein folding burden on ER while, on the other hand, enhances ATF4 translation likely to restore protein homeostasis through transcriptional control. ATF4 is a transcription factors known to regulate the expression of several genes involved in not only protein homeostasis but also neuroplasticity, glucose and lipid homeostasis. Since we found that 2-DG led increase in plasticity gene program and LTP is transcription dependent and we also found that 2-DG led to upregulation of around 300 ATF4 target genes, we hypothesized that 2-DG led increase in the expression of Bdnf and likely other plasticity genes is mediated by ATF4. Firstly, we tested if 2-DG has direct effect on ATF4 binding to the promoters of its target genes. For this, we expressed Trib3 promoter with or without mutation in ATF4 binding site tagged with luciferase in primary neurons and assessed if 2-DG led induction in Trib3 promoter activity is blocked with ATF4 binding site mutation or not. Of note, we found that ATF4 binding site mutation completely blocked the 2-DG led induction of Trib3 promoter activity indicating that 2-DG has a direct effect on ATF4 binding to the promoters of its target genes (FIG. 7N). Next, we examined if ATF4 component of eIF2α phosphorylation is necessary with respect to 2-DG led increase in the expression of Bdnf and likely other plasticity genes or not. For this, we knocked down ATF4 using adenoviral expression of dominant negative ATF4 (ATF4ARK, which lack ATF4 DNA binding domain) in primary neurons. Importantly, we found that ATF4 knockdown blocked 2-DG led increase in Bdnf gene expression (FIG. 70). Next, we tested if ATF4 is sufficient in upregulating Bdnf gene expression or not. To this end, we overexpressed adenoviral construct of ATF4 in primary neurons for 24 h. The significant upregulation of the expression of ATF4 target genes, Chac1 and Trib3 confirmed the expression of ATF4 in primary neurons (FIGS. 16A and 16B). Interestingly, ATF4 expression was sufficient in significantly inducing Bdnf gene expression (FIG. 7P).

Next, we tested if 2-DG could induce the paradoxical increase in ATF4 translation or not. To this end, we overexpressed 5′UTR ATF4 luciferase reporter using AAV8 viral vector in mouse brain and then injected 2-DG (10 mg/Kg) intraperitoneally for 4 h. We found a significant increase in ATF4 translation in the mouse brain in response to 2-DG treatment indicating that 2-DG effectively engages ATF4 signaling cascade in the brain (FIG. 7Q). These findings suggested that N-linked glycosylation-UPR-eIF2α phosphorylation-ATF4 signaling cascade of ER stress is a necessary glucose sensing axis of Bdnf and likely other plasticity genes and is also sufficient in inducing the plasticity gene program (FIG. 7Q).

### ATF4 Directly Regulates Bdnf and Other Plasticity Genes

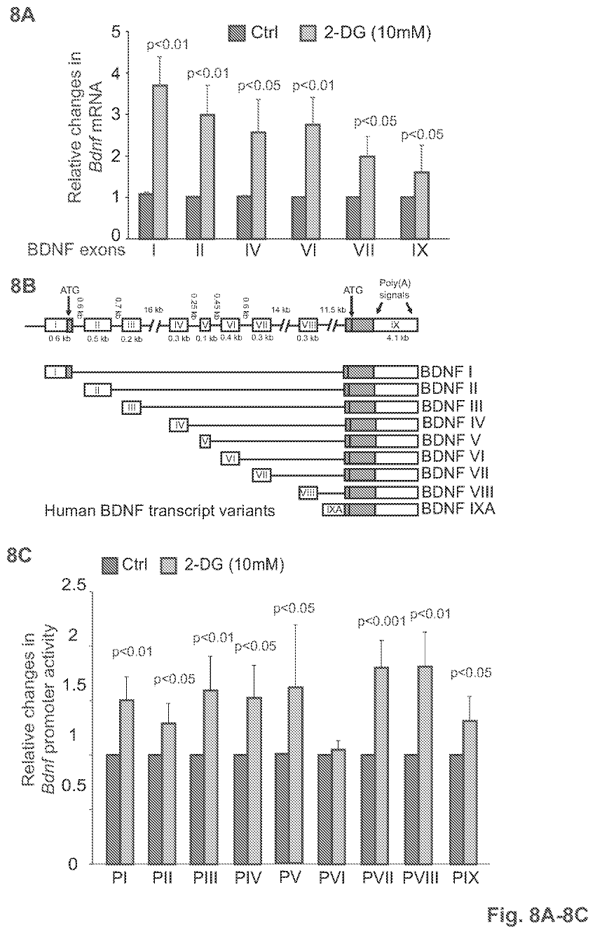

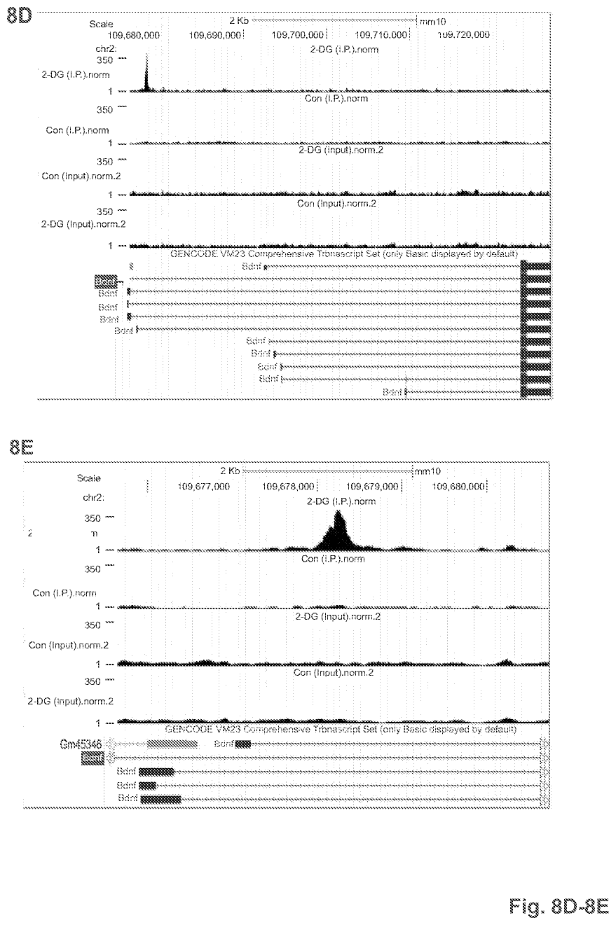

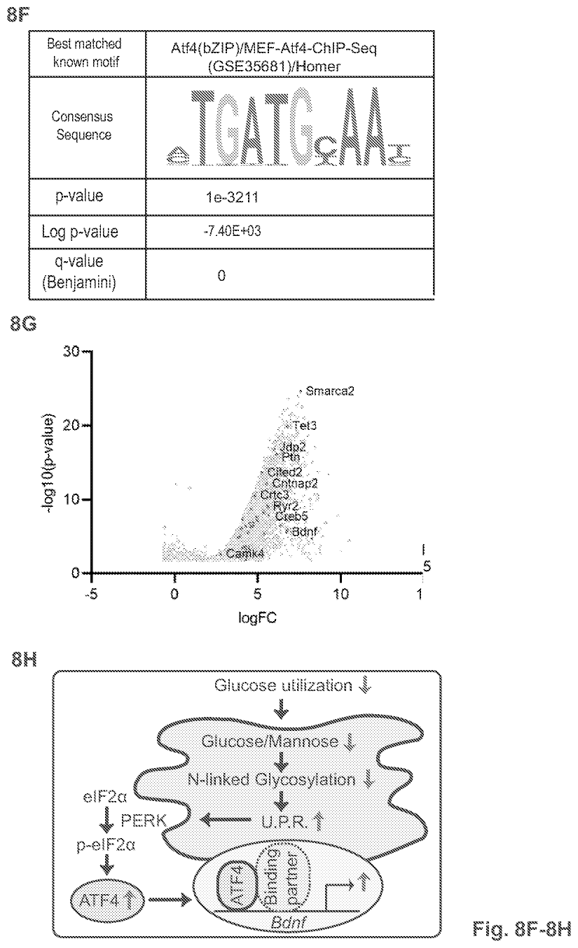

Bdnf gene is transcribed as multiple transcript variants in mouse and human. Using specific primers for each transcript variant, we tested six endogenous transcript variants of Bdnf to see which one gets upregulated in response to 2-DG treatment in mouse primary neurons. We found that all transcript variants were upregulated with 2-DG treatment (FIG. 8A). Each exon of Bdnf is expressed as a separate transcript variant and has its own promoter. However, coding region of these different transcript variants eventually translate into one functional BDNF protein. Next, we identified the response of different transcript variants or promoters of Bdnf to 2-DG treatment. To this end, we expressed nine different human Bdnf promoters each tagged with luciferase in murine hippocampal cell line, HT22 cells (FIG. 8B) and examined the promoter activity in response to 2-DG treatment. We found that all Bdnf promoters except Bdnf promoter VI were significantly upregulated in response to 2-DG treatment (FIG. 8C). Next, we examined if ATF4 directly binds to Bdnf promoter or not. For this, we performed chromatin immunoprecipitation using antibody against ATF4 in response to 2-DG treatment in primary neurons. We found that 2-DG treatment led to a selective enrichment of ATF4 close to the transcription start site of Bdnf (FIGS. 8D and 8E). We, next, ran de novo motif analysis that returns most enriched sequences within the peak region. This analysis returned ATF4 as the most significant motif, confirming the CHIP specificity (FIG. 8F). ATF4 is a bZIP family transcription factor that needs to either homodimerize by itself or heterodimerize with another binding partner in order to bind to target genes (Fujii et al., 2000). Interestingly, de novo motif analysis also returned other enriched sequences such as Chop, AARE, CEBP:AP1, CEBPA, Ddit3::Cebpa, CEBPB, JUN, and JUN (Var.2) etc. within the peak region indicating these as potential binding partners with the ATF4. Next, we asked if 2-DG treatment increases binding of ATF4 with only Bdnf or other known plasticity genes as well. Interestingly, we found that 2-DG treatment led to enhanced binding of ATF4 to several plasticity genes known to play important roles in learning and memory, repair, neurogenesis and neuronal survival (FIG. 8G). Altogether, these data suggest direct binding of ATF4 with Bdnf and several other plasticity genes by either homodimerizing with itself or heterodimerizing with other potential binding partners in response to 2-DG treatment (FIG. 8H).

### Discussion

The current findings provide clear evidence that a calorie restriction mimetic, 2-DG, normalizes learning and memory deficit in transgenic mouse model of chronic neurodegenerative disorder, Alzheimer disease and improves sensory and motor functions in mice models of ischemic and hemorrhagic stroke. Previous clinical studies have shown that calorie restriction and intermittent fasting improve learning and memory in many clinical studies (Horie et al., 2016; Leclerc et al., 2020; Ooi et al., 2020; Smith et al., 2010; Witte et al., 2009). Moreover, calorie restriction has also been shown to improve functional outcome following ischemic stroke in animal models (Ciobanu et al., 2017; Huang et al., 2021; Yu and Mattson, 1999). Our current findings demonstrating 2-DG as a calorie restriction mimetic move the field forward by removing the major clinical limitation of putting human being with a stressful phase of diet restriction which may also compromise their nutritional balance. Additionally, the dosage of 2-DG can be easily standardized which is a big challenge with calorie restriction diets.

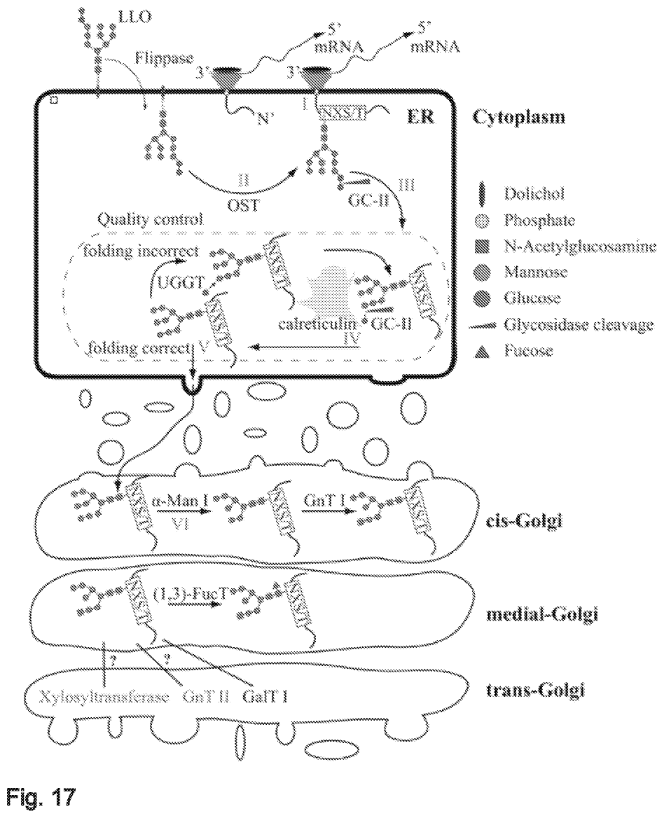

Moreover, we unraveled the mechanism of action of 2-DG by which it provides the beneficial effects in AD and stroke. Since glucose is the major energy substrate of neurons, the energy deficit is an immediate consequence of diminished glucose uptake and utilization with 2-DG (FIG. 4A). Interestingly, our high throughput RNA sequencing data didn't show any significant change in mitochondrial gene signature in response to 2-DG treatment indicating that the compensatory adaptive response of neurons to 2-DG led energy deficit likely involves transcription independent signaling. Of note, we found a remarkably strong gene signature for ER associated unfolded protein response (FIG. 6 and FIG. 12), which is process activated to restore the protein homeostasis within ER (Frakes and Dillin, 2017). It is likely that there is a tradeoff for glucose between energy production and N-linked glycosylation needed for proper protein folding under diminished glucose condition. Since neurons are energy intensive cells, under 2-DG led diminished intracellular glucose condition, most of the available intracellular glucose along with other energy substrates such as amino acids, lactate, ketone bodies or fatty acids are likely prioritized towards energy production to keep the energy supply unimpeded. This prioritization of glucose for energy production under 2-DG led energy stress condition would lead to diminished glucose availability for N-linked glycosylation that in turn leads to a compensatory activation of adaptive program in the form of UPR along with activation of downstream signaling cascade such as eIF2α phosphorylation through PERK activation, paradoxical increase in ATF4 translation, and increase in transcriptional activity of ATF4 leading to an increase in expression of Bdnf and likely other plasticity genes (FIGS. 7 and 8; FIGS. 15-17) to support a transcription dependent long term potentiation (FIG. 4), learning and memory (FIG. 2), and cell survival and repair program (FIG. 3). Importantly, contrary to the classical thinking of UPR with always negative consequences, diminished N-linked glycosylation and consequent activation of UPR was recently found to improve mitochondrial functioning by promoting respiratory chain super-complexes through PERK-eIF2α-ATF4 axis (Balsa et al., 2019). This finding along with ours indicate that 2-DG led decrease in N-linked glycosylation and consequent UPR activation induces two simultaneous adaptive programs 1.) improving the mitochondrial function and 2.) upregulating plasticity gene program required for improving learning and memory and cell survival and repair program through a downstream master metabolic transcriptional regulator, ATF4.

UPR triggers several downstream signaling cascades to restore protein homeostasis. For instance, UPR triggers eIF2α phosphorylation through PERK activation, which, on one hand, suppresses general protein translation to diminish the substrate burden on chaperones engaged in restoring the protein homeostasis and, on other hand, enhances the paradoxical translation of specific mRNAs such as ATF4 that regulates the transcription of a cassette of genes involved in restoration of protein homeostasis and thereby lessens the stress condition (Trinh et al., 2012; Wang et al., 2020). It has been shown by different groups that de novo protein synthesis is necessary for the long term memory formation (Alberini, 2008; Costa-Mattioli et al., 2005; Santini et al., 2014). Several earlier studies have implicated eIF2α phosphorylation as a key regulatory factor impacting synaptic plasticity and long term memory formation by affecting general translation (Costa-Mattioli et al., 2005; Costa-Mattioli et al., 2007; Jiang et al., 2010; Ma et al., 2013; Sidrauski et al., 2013). Importantly, activation of ATF4 signaling cascade downstream of eIF2α phosphorylation has also been shown to play important role in synaptic plasticity, long-term memory formation and cell survival (Pasini et al., 2015; Sun et al., 2018). We found that 2-DG leads to a mild upregulation of eIF2α phosphorylation (FIGS. 6E and 7G) and a significant upregulation of ATF4 transcriptional activity evident by enhanced expression of ATF4 target genes, Trib3 and Chac1 (FIGS. 6F and 6G). Interestingly, we found that ATF4 was necessary as well as sufficient in 2-DG led increase in expression of Bdnf (FIGS. 70 and 7P) and likely other plasticity genes (FIG. 8G). Bdnf is one of the plasticity genes that has been shown to play important roles in synaptic plasticity, learning and memory, neurogenesis and neuronal survival (Kowianski et al., 2018; Leal et al., 2017). Additionally, as ATF4 is a critical anabolic transcriptional regulator responsible for regulation of genes involved in amino-acid synthesis and mobilization, its activation likely drives the mobilization of amino acids such as cysteine, glycine and glutamate towards glutathione synthesis (Ratan et al., 1994) or glutamate and other amino acids towards anaplerotic support of TCA cycle (Rink et al., 2017; Zaghmi et al., 2020) for fulfilling the energy needs away from protein synthesis as required for the cell survival under stress conditions (FIGS. 9A and 9B) and therefore, could play important role in 2-DG led improvement in neuronal survival, and thereby, in learning and memory in AD and functional recovery following stroke. Increasing the protein synthesis under stress condition might not be beneficial as it will further increase the load on ER which is already engaged in resolving the buildup of misfolded proteins by inducing the UPR signaling. Altogether, our findings indicate that induction of ATF4 transcriptional activity downstream of eIF2α phosphorylation is essential and sufficient in 2-DG led increase in the expression of Bdnf and likely other plasticity genes that play important role in improving synaptic plasticity, learning and memory, cell survival, and cell repair programs.

Our finding that 2-DG increases Bdnf gene expression in various in-vitro and in-vivo models such as mouse primary cortical neurons, human i.p.s. derived primary cortical neurons, mouse brain cortex and hippocampus in response to 2-DG treatment is corroborated well with some findings from other groups where calorie mimetic 2-DG, as well as calorie restriction diet regimes, were found to increase expression of Bdnf (Kishi et al., 2015; Lee et al., 2000; Yao et al., 2011). But interestingly, 2-DG was contrarily found to inhibit the Bdnf gene expression in JTC-19 lung cancer line and also HEK293 cell line (Garriga-Canut et al., 2006).

We found that neurons upregulate not just Bdnf but a broad cassette of plasticity genes (FIG. 6A). In their hippocampal studies, they injected rats intraperitoneally with 250 mg/kg/day 2-DG for two weeks while our treatments included either slow delivery of 2-DG administered through intracerebroventricular infusion by Alzet mini-osmotic pump at (1 mg/Kg/day) or intraperitoneal injection of 10 mg/kg/day 2-DG for 3-4 weeks in mice starting 24 h after stroke. These doses are much lower as compared to their doses, which could be a reason behind opposite results, although it is not clear.

The prospect of using 2-DG as a therapeutic drug against AD patient in the clinical setting is interesting since it can easily cross the blood brain barrier (indicated by FDG PET studies), and the safe doses of 2-DG as an anticancer agent have been assessed in patients through phase I/II clinical trial (Raez et al., 2013; Stein et al., 2010). We found that intraperitoneal injection of 2-DG at 10 mg/Kg or intracerebroventricular infusion of 2-DG at 1 g/Kg/day was sufficient in driving the adaptive plasticity program. The respective equivalent doses in human calculated on the basis of body surface area as per FDA guideline would be around 1 mg/Kg or 100 μg/Kg/day (Nair and Jacob, 2016), which are much lower than the recommended safe doses of 2-DG for human patients (Raez et al., 2013; Stein et al., 2010). Although the efficacy of 2-DG for the improvement in cognitive decline in AD patients requires a proper clinical study, our study suggests that this dose of 2-DG should not induce hypoglycemia addressing the common concern of not interfering with the glucose metabolism as it is critical for the brain function.

### Key Resources Table:

| | | |
|---|---|---|
| REAGENT or RESOURCE | SOURCE | IDENTIFIER |

Antibodies

| | | |
|---|---|---|
| Anti-Glut3 | Abcam | Cat#ab41525; |
| | | RRID: |
| | | AB_732609 |
| Anti-ß-actin | Sigma-Aldrich | Cat#A5316; |
| | | RRID: |
| | | AB_476743 |
| Anti-BDNF (N-20) | Santa Cruz | Cat#sc-546; |
| | Biotechnology | RRID: |
| | | AB_630940 |
| Anti-α-tubulin | Sigma-Aldrich | Cat#T8203; |
| | | RRID: |
| | | AB_1841230 |
| Anti-phospho-AMPKα (Thr 172) | Cell Signaling | Cat#2535; |
| | Technology | RRID: |
| | | AB_331250 |
| Anti-AMPKα | Cell Signaling | Cat#2532; |
| | Technology | RRID: |
| | | AB_330331 |
| Anti-GFP | Cell Signaling | Cat#2555; |
| | Technology | RRID: |
| | | AB_10692764 |
| Anti-phospho-EIF2S1 (S51) | Abcam | Cat#ab32157; |
| | | RRID: |
| | | AB_732117 |
| Anti-eIF2α | Cell Signaling | Cat#9722S; |
| | Technology | RRID: |
| | | AB_2230924 |
| Anti-ß-actin | Sigma-Aldrich | Cat#A5316; |
| | | RRID: |
| | | AB_476743 |

Bacterial and Virus Strains

| | | |
|---|---|---|
| Ad-CMV GFP | ViraQuest, Inc. | MSRN: 22576 |
| | (North liberty, IA) | |
| Ad-AMPKα1 D.N. | ViraQuest, Inc. | MSRN: 17930 |
| | (North liberty, IA) | |
| AAV8-EGFP-iCre | Vector Biolabs | 7097 |
| AAV8-GFP | Vector Biolabs | 7061 |
| Ad-mATF4 d-RK | ViraQuest, Inc. | 25195 |
| | (North liberty, IA) | |
| Ad-mATF4 WT | ViraQuest, Inc. | 22902 |
| | (North liberty, IA) | |

Biological Samples

| | | |
|---|---|---|
| Primary cortical neurons from CD-1 strain mice | Charles River | See |
| | | experimental |
| | | models' section |
| Human iPSC line (C1-1) was previously generated | See (Wen et al. | See |
| from skin biopsy samples of male newborn and had | 2014) | experimental |
| been fully characterized and passaged on MEF feeder | | models' section |
| layers | | |
| C57BL/6 mice | Charles River | See |
| | | experimental |
| | | models' section |
| C57BL/6J mice | Jackson's | Stock |
| | laboratory | number#000664 |
| 5xFAD mice | Jackson's | Stock |
| | laboratory | Number#034848- |
| | | JAX |
| Ppp1R15bflox/flox mice | Generous gifted | See |
| | from Ann-Hwee | experimental |
| | Lee (Regeneron | models' section |
| | Pharmaceuticals) | |

Chemicals, Peptides, and Recombinant Proteins

| | | |
|---|---|---|
| 2-Deoxyglucose | Sigma-Aldrich | Cat#D6134; |
| | | CAS Number |
| | | 154-17-6 |
| Collagenase Type VII-S | Sigma-Aldrich | Cat#C2399; |
| | | CAS Number |
| | | 9001-12-1 |
| Diluent Sodium chloride (0.9% solution) | Hospira | Cat#00409488820 |
| Metformin | Sigma-Aldrich | Cat#D150959; |
| | | CAS Number |
| | | 1115-70-4 |
| Actinomycin D | Sigma-Aldrich | Cat#AA1410; |
| | | CAS Number |
| | | 50-76-0 |
| Tunicamycin | Sigma-Aldrich | Cat#T7765; |
| | | CAS Number |
| | | 11089-65-9 |
| GSK2606414 (PERK inhibitor I) | | Cat#516535; |
| | | CAS Number |
| | | 1337531-89-1 |
| 2-Mercaptoethanol | Sigma-Aldrich | Cat#M3148; |
| | | CAS Number |
| | | 60-24-2 |
| 1,4-Dithiothreitol (DTT) | ThermoFisher | Cat#R0861; |
| | Scientific | CAS Number |
| | | 3483-12-3 |
| Triton X-100 | Sigma-Aldrich | Cat#X100; CAS |
| | | Number 9002- |
| | | 93-1 |
| Protease Inhibitor Cocktail | Sigma-Aldrich | Cat#P8340 |
| Pheylmethanesulfonyl fluoride (PMSF) | Sigma-Aldrich | Cat#P8340; |
| | | CAS Number |
| | | 329-98-6 |
| Ethylene glycol-bis(2aminoehylether)-N, N, N, N- | Sigma-Aldrich | Cat#E3889; |
| tetra acetic acid (EGTA) | | CAS Number |
| | | 67-42-5 |
| Trizma base | Sigma-Aldrich | Cat#T4661; |
| | | CAS Number |
| | | 77-86-1 |
| Sodium Chloride | Sigma-Aldrich | Cat#S3014; |
| | | CAS Number |
| | | 7647-14-5 |
| Potassium chloride | Sigma-Aldrich | Cat#P5405; |
| | | CAS Number |
| | | 7447-40-7 |
| Sodium phosphate dibasic | Sigma-Aldrich | Cat#S5136; |
| | | CAS Number |
| | | 7558-79-4 |
| Sodium bicarbonate | Sigma-Aldrich | Cat#S6014; |
| | | CAS Number |
| | | 144-55-8 |
| D-Glucose | Sigma-Aldrich | Cat#G8270; |
| | | CAS Number |
| | | 50-99-7 |
| D-Mannose | Sigma-Aldrich | Cat#M6020; |
| | | CAS Number |
| | | 3458-28-4 |
| complete Mini EDTA-free (Protease inhibitor | Roche | Cat#11836170001 |
| cocktail) | | |
| 16% Formaldehyde, Methanol Free | Cell Signaling | Cat#12606S |
| | Technology | |
| Glycine | Sigma-Aldrich | |
| MG132 | Sigma-Aldrich | Cat#M7449; |
| | | CAS |
| | | Number: 13340 |
| | | 7-82-6 |
| UltraPure 1M Tris-HCl, pH 8.0 | Invitrogen | Cat#15568-025 |
| UltraPure 0.5 M EDTA, pH 8.0 | Invitrogen | Cat#15575-038 |
| UltraPure 10% SDS | Invitrogen | Cat#15553-035 |
| Dynabeads Protein G | Invitrogen | Cat#10003D |
| 5M Sodium chloride solution | Sigma-Aldrich | Cat#59222C |
| Sodium-Deoxycholate | ThermoFisher | Cat#89904 |
| | Scientific | |
| Lithium chloride solution 8M | Sigma-Aldrich | Cat#L7026 |
| NP-40 | ThermoFisher | Cat#85124 |
| | Scientific | |

Critical Commercial Assays

| | | |
|---|---|---|
| Nucleospin RNA Kit | TaKaRa | Cat#740955.250 |
| MagMAX mirVana Total RNA Isolation Kit | ThermoFisher | A27828 |
| | Scientific | |
| DC Protein Assay Kit 1 | Bio-Rad | 5000111 |
| Live/dead kit | ThermoFisher | L3224 |
| | Scientific | |
| Dual-Luciferase Reporter 1000 Assay System | Promega | E1980 |
| Taqman RNA-to Ct 1-Step kit | ThermoFisher | 4392938 |
| | Scientific | |
| Tagment DNA Enzyme and Buffer | Illumina | 20034210 |
| SuperScipt III First Strand Synthesis System | Invitrogen | 18080-051 |
| for RT PCR | | |
| SYBR Green Master Mix | Applied | Cat#4309155 |
| | Biosystems | |
| AMPureXP | Beckman Coulter | Cat#A63880 |
| Fast Ion Plasmid Maxi kit | IBI Scientific | Cat#IB47125 |

Experimental Models: Cell Lines

| | | |
|---|---|---|
| Mouse HT-22 Neuroblast Cell Line | EMD Millipore | Cat#SCC129 |
| Oligonucleotides | | |
| FAM labeled Crebl (Taqman probe) | ThermoFisher | Mm00501607_ |
| | Scientific | m1 |
| FAM labeled Bdnf (Taqman probe) | ThermoFisher | Mm04230607_ |
| | Scientific | s1 |
| FAM labeled Rbbp4 (Taqman probe) | ThermoFisher | Mm00771401_ |
| | Scientific | g1 |
| FAM labeled Chacl (Taqman probe) | ThermoFisher | Mm00509926_ |
| | Scientific | m1 |
| FAM labeled Trib3 (Taqman probe) | ThermoFisher | Mm00454879_ |
| | Scientific | m1 |
| FAM labelled Ddit3 (Taqman probe) | ThermoFisher | Mm01135937_ |
| | Scientific | g1 |
| FAM labelled Bip (Taqman probe) | ThermoFisher | Mm00517691_ |
| | Scientific | m1 |
| Mouse ACTB Control Mix | ThermoFisher | 4351315 |
| | Scientific | |
| BDNF IV-F (GTGAGGTTTGTGTGGACCCC) | ThermoFisher | N/A |
| (SEQ ID NO: 1) | Scientific | |
| BDNF IV-R (ATTGGGCCGAACTTTCTGGT) | ThermoFisher | N/A |
| (SEQ ID NO: 2) | Scientific | |
| CREBa-F (GGCTCCAGATTCCATGGTC) (SEQ ID | ThermoFisher | N/A |
| NO: 3) | Scientific | |
| CREBa-R (GTGTTACGTGGGGGAGAGAA) (SEQ | ThermoFisher | N/A |
| ID NO: 4) | Scientific | |
| CREBb-F (GGCTCCAGATTCCATGGTC) (SEQ ID | ThermoFisher | N/A |
| NO: 5) | Scientific | |
| CREBb-R (GTGTTACGTGGGGGAGAGAA) (SEQ | ThermoFisher | N/A |
| ID NO: 6) | Scientific | |
| GAPDH-F (TGGTCTCCTCTGACTTCAACAGCG) | ThermoFisher | N/A |
| (SEQ ID NO: 7) | Scientific | |
| GAPDH-R | ThermoFisher | N/A |
| (AGGGGTCTACATGGCAACTGTGAG) (SEQ ID | Scientific | |
| NO: 8) | | |
| BDNF I-F (TTGAAGCTTTGCGGATATTGCG) | ThermoFisher | N/A |
| (SEQ ID NO: 9) | Scientific | |
| BDNF I-R (AAGTTGCCTTGTCCGTGGAC) (SEQ | ThermoFisher | N/A |
| ID NO: 10) | Scientific | |
| BDNF II-F | ThermoFisher | N/A |
| (TGGTATACTGGGTTAACTTTGGGAAA) (SEQ | Scientific | |
| ID NO: 11) | | |
| BDNF II-R (AAGTTGCCTTGTCCGTGGAC) (SEQ | ThermoFisher | N/A |
| ID NO: 12) | Scientific | |
| BDNF IV-F 2 | ThermoFisher | N/A |
| (GAAATATATAGTAAGAGTCTAGAACCTTG) | Scientific | |
| (SEQ ID NO: 13) | | |
| BDNF IV-R 2 (AAGTTGCCTTGTCCGTGGAC) | ThermoFisher | N/A |
| (SEQ ID NO: 14) | Scientific | |
| BDNF VI-F (GCTTTGTGTGGACCCTGAGTTC) | ThermoFisher | N/A |
| (SEQ ID NO: 15) | Scientific | |
| BDNF VI-R (AAGTTGCCTTGTCCGTGGAC) | ThermoFisher | N/A |
| (SEQ ID NO: 16) | Scientific | |
| BDNF VII-F | ThermoFisher | N/A |
| (CCTGAAAGGGTCTGCGGAACTCCA) (SEQ ID | Scientific | |
| NO: 17) | | |
| BDNF VII-R | ThermoFisher | N/A |
| (GAAGTGTACAAGTCCGCGTCCTTA) (SEQ ID | Scientific | |
| NO: 18) | | |
| BDNF IX-F | ThermoFisher | N/A |
| (GGACTATGCTGCTGACTTGAAAGGA) (SEQ | Scientific | |
| ID NO: 19) | | |
| BDNF IX-R | ThermoFisher | N/A |
| (GAGTAAACGGTTTCTAAGCAAGTG) (SEQ ID | Scientific | |
| NO: 20) | | |
| GAPDH-F 2 (ATGGTGAAGGTCGGTGTGAACG) | ThermoFisher | N/A |
| (SEQ ID NO: 21) | Scientific | |
| GAPDH-R 2 (CGCTCCTGGAAGATGGTGATGG) | ThermoFisher | N/A |
| (SEQ ID NO: 22) | Scientific | |

Recombinant DNA

| | | |
|---|---|---|
| Trib3 promoter reporter | Generated in the | See Lange et al. |
| | lab | 2008, JEM |
| Mutant Trib3 promoter reporter lacking 33-bp ATF4 | Generated in the | See Lange et al. |
| binding site | lab | 2008, JEM |
| pGL4.15 hBDNF I promoter reporter | Generous gift from | See Pruunsild et |
| | Dr. Tõnis | al. 2011, J. |
| | Timmusk | Neurosci. |
| pGL4.15 hBDNF II promoter reporter | Generous gift from | See Pruunsild et |
| | Dr. Tõnis | al. 2011, J. |
| | Timmusk | Neurosci. |
| pGL4.15 hBDNF III promoter reporter | Generous gift from | See Pruunsild et |
| | Dr. Tõnis | al. 2011, J. |
| | Timmusk | Neurosci. |
| pGL4.15 hBDNF IV promoter reporter | Generous gift from | See Pruunsild et |
| | Dr. Tõnis | al. 2011, J. |
| | Timmusk | Neurosci. |
| pGL4.15 hBDNF V promoter reporter | Generous gift from | See Pruunsild et |
| | Dr. Tõnis | al. 2011, J. |
| | Timmusk | Neurosci. |
| pGL4.15 hBDNF VI promoter reporter | Generous gift from | See Pruunsild et |
| | Dr. Tõnis | al. 2011, J. |
| | Timmusk | Neurosci. |
| pGL4.15 hBDNF VII promoter reporter | Generous gift from | See Pruunsild et |
| | Dr. Tõnis | al. 2011, J. |
| | Timmusk | Neurosci. |
| pGL4.15 hBDNF VIII promoter reporter construct | Generous gift from | See Pruunsild et |
| | Dr. Tõnis | al. 2011, J. |
| | Timmusk | Neurosci. |
| pGL4.15 hBDNF IX promoter reporter construct | Generous gift from | See Pruunsild et |
| | Dr. Tõnis | al. 2011, J. |
| | Timmusk | Neurosci. |
| pRL-TK-renilla luciferase reporter plasmid | Promega | Cat#E2241 |

Software and Algorithms

ANY-maze 5.1
Signal (Version 4.11)
Graphpad Prism 9
Nikon Digital Sight DS-L3
Adobe Illustrator CS6
Adobe Photoshop CS6

Other

| | | |
|---|---|---|
| Fetal bovine serum | Life Technologies | Cat#16140-071 |
| Horse serum | Life Technologies | Cat#26050-088 |
| Penicillin-Streptomycin | Life Technologies | Cat#15140122 |

### Materials and Methods

### Animal Models

All animal procedures were approved by the Weill Cornell Medicine Institutional Animal Care and Use Committee (Animal protocol: 2014-0029) and conducted in accordance with the NIH Guide for the Care and Use of Laboratory Animals and ARRIVE guidelines. We used C57/BL6 mice for the induction of ischemic stroke and hemorrhagic stroke. 8-10 weeks old female pregnant CD1 mice were used for primary neuronal culture. Ppp1R15b^{flox/flox} mice were obtained from Ann-Hwee Lee's group currently working at Regeneron and were bred at Burke Neurological Institute. The genotypes of Ppp1R15b^{flox/flox} mice were assessed with PCR using DNA isolated from clipped tails of mice. Twelve weeks old male Ppp1R15b^{flox/flox} mice were used for inactivation of R15b regulatory subunit of protein phosphatase 1 in the hippocampus through AAV8 GFP or Cre intracranial injection. In the current study, 5×FAD mouse was used as a model of AD as this model represents pathological characteristics seen in both genetic as well as sporadic forms of AD. 5×FAD mice (B6/SJL genetic background) was purchased from Jackson's laboratory and was maintained by breeding male heterozygous transgenic mice with wild type female mice. The 5×FAD mice express human APP and PSEN1 transgenes with a total of five AD-linked mutations: the Swedish (K670N/M671L), Florida (I716V), and London (V717I) mutations in APP, and the M146L and L286V mutations in PSEN1. In the current study, 6-7 months old 5×FAD mice were used for the Alzet mini osmotic pump implantation followed by memory assessment, LTP assessment, and gene expression studies.

### Mouse Primary Immature Cortical Neuronal Culture

Primary immature neuronal cultures were prepared from cerebral cortex of E14 embryos (day 14 of gestation) of CD1 mice under sterile conditions. Dissected cortices were incubated at 37° C. for 20 min in EBSS solution containing papain, L-cystine and EDTA, which was activated at 37ºC for 30 min and then was filtered through 22-micron filter (Millipore, catalog number—SCGP00525). Cortices were then mechanically dissociated by trituration with glass pipettes. The rDNase was added in dissociated cells followed by incubation of these dissociated cells at 37ºC for 3 min. Thereafter, these cells were centrifuged at 2800 rpm for 5 min. Then supernatant was discarded, and cell were resuspended gently in a 5 ml mixture of EBSS and 10/10 solution (4:1 ratio). Then 5 ml of 10/10 solution was added gently on top and these resuspended cells were centrifuged at 2800 rpm for 10 min. Supernatant was again discarded and cells were resuspended in 10 ml MEM, Glutamax supplement (Thermo Fisher Scientific, Catalog number—41090036) containing 10% heat inactivated fetal bovine serum (Thermo Fisher Scientific, Catalog number—16140071), 5% heat inactivated horse serum (Thermo Fisher Scientific, Catalog number—26050088), and 1% penicillin/streptomycin (Thermo Fisher Scientific, Catalog number—15140122). Thereafter, resuspended cells were filtered through 70-micron cell strainer (Thomas Scientific, catalog number—4620F02) followed by their counting through Hemocytometer. Cells were, finally, plated at the density of 1 million cells/ml onto poly-D-Lysine (PDL; Sigma Aldrich, catalog number—P6407-10X5MG) coated pates and placed in CO₂-buffered incubators at 37° C.

Culture of Human iPSCs and Differentiation into Cortical Neurons

Human iPSC line (C1-1) was previously generated from skin biopsy samples of male newborn and had been fully characterized and passaged on MEF feeder layers (Wen et al. 2014). All studies followed institutional IRB and ISCRO protocols approved by University of Pennsylvania Perelman School of Medicine. Human iPSCs were differentiated into cortical neurons following the previously established protocol (Wen et al. 2014). Briefly, hiPSCs colonies were detached from the feeder layer with 1 mg/ml collagenase (Thermo Fisher Scientific) treatment for 30 min and suspended in embryonic body (EB) medium, consisting of bFGF-free iPSC medium supplemented with 2 μM Dorsomorphin (Tocris) and 2 μM A-83 (Tocris), in non-treated polystyrene plates for 4 days with a daily medium change. After 4 days, EB medium was replaced by neural induction medium (NPC medium) consisting of DMEM/F12 (Thermo Fisher Scientific), 1×N2 supplement (Thermo Fisher Scientific), 1×MEM NEAA (Thermo Fisher Scientific), 2 μg/ml heparin (Sigma) and 2 μM cyclopamine (Tocris). The floating EBs were then transferred to Matrigel (Corning)-coated 6-well plates at day 7 to form neural tube-like rosettes. The attached rosettes were kept for 15 days with NPC medium change every other day. On day 22, the rosettes were picked mechanically and transferred to low attachment plates (Corning) to form neurospheres in NPC medium containing 1×B27 (Thermo Fisher Scientific). The neurospheres were then dissociated with Accutase (Thermo Fisher Scientific) and placed onto Poly-D-Lysine/laminin (Sigma)-coated coverslips in the neuronal culture medium, consisting of Neurobasal medium (Thermo Fisher Scientific) supplemented with 1× Glutamax (Thermo Fisher Scientific), 1×B27 (Thermo Fisher Scientific), 1 μM cAMP (Sigma), 200 ng/ml L-Ascorbic Acid (Sigma), 10 ng/ml BDNF (PeproTech) and 10 ng/ml GDNF (PeproTech). Half of the medium was replaced once a week during continuous culturing.

### In-Vitro Plasmid Transfection and Adenoviral Transduction

Mouse primary neurons were transduced with adenoviral constructs of GFP (Ad-CMVGFP), AMPK D.N. (Ad-AMPKα1 D.N.), ATF4 (Ad-mATF4 WT) or ATF4dRK (Ad-mATF4 d-RK) at 200MOI for 4 h in HBSS (Thermo Fisher Scientific, Catalog number—14025134). Thereafter, HBSS was replaced with MEM, Glutamax supplement containing 10% heat inactivated fetal bovine serum, 5% heat inactivated horse serum, and 1% penicillin/streptomycin. Primary neurons transduced with adenoviral constructs of either AMPK D.N. or ATF4 dRK and their respective controls were incubated in this media for 72 h and then treated with 2-DG for a specified time before being harvested for the experiments. Primary neurons transduced with adenoviral construct of ATF4 and its respective control, GFP were incubated for 24 h and then cells were harvested for the gene expression study.

### Cell Viability Assay:

Cell viability of mouse primary neurons treated with increasing concentrations of 2-DG (1 mM-15 mM) for 6 h was assessed by calcein-acetoxymethyl ester (AM)/ethidium homodimer-1 staining using live/dead viability/cytotoxicity kit (Thermo Fisher Scientific, Catalog number—L3224) using epifluorescence microscopy with inverted microscope Nikon ECLIPSE TS100 attached with digital capture system Nikon digital sight DS-L3.

### Non-Targeted Metabolic Profiling and Metabolic Pathway Analysis

Targeted Metabolite profiling was performed according to a method described in a previous publication (Chen et al., 2016). Polar metabolites were extracted using cold 80% methanol. The extracts were dried completely with a Speedvac and redissolved in water before it was applied to the hydrophilic interaction chromatography LC-MS. The sample injection order was randomized. Metabolites were measured on a Q Exactive Orbitrap mass spectrometer (Thermo Scientific), which was coupled to a Vanquish UPLC system (Thermo Scientific) via an Ion Max ion source with a HESI II probe (Thermo Scientific). A Sequant ZIC-PHILIC column (2.1 mm i.d.×150 mm, particle size of 5 μm, Millipore Sigma) was used for separation of metabolites. A 2.1×20 mm guard column with the same packing material was used for protection of the analytical column. Flow rate was set at 150 L/min. Buffers consisted of 100% acetonitrile for mobile phase A, and 0.1% NH₄OH/20 mM CH₃COONH₄ in water for mobile phase B. The chromatographic gradient ran from 85% to 30% A in 20 min followed by a wash with 30% A and re-equilibration at 85% A. The column temperature was set to 30° C. and the autosampler temperature was set to 4° C. The Q Exactive was operated in full scan, polarity-switching mode with the following parameters: the spray voltage 3.0 kV, the heated capillary temperature 300° C., the HESI probe temperature 350° C., the sheath gas flow 40 units, the auxiliary gas flow 15 units. MS data acquisition was performed in the m/z range of 70-1,000, with 70,000 resolution (at 200 m/z). The AGC target was 3,000,000 and the maximum injection time was 100 ms. The MS data was processed using XCalibur 4.1 (Thermo Scientific) to extract the metabolite signal intensity for relative quantitation. Metabolites were identified using an in-house library established using chemical standards. Identification required exact mass (within 5 ppm) and standard retention times. Metabolic pathway enrichment analysis and pathway topology analysis were conducted using MetaboAnalyst 3.0 computational platform (Xia et al., 2015). In pathway enrichment analysis, a single P value is calculated for each metabolic pathway. Pathway topology analysis measured the significance of a given experimentally identified metabolite in a pre-defined metabolic pathway.

### Cell Based Luciferase Assays

Primary neurons were co-transfected with various plasmids such as different Bdnf exon promoters each tagged with firefly luciferase or Trib3 promoter tagged with firefly luciferase and pTK-renilla luciferase plasmid at 1:10 ratio using Lipofectamine 2000 (Thermo Fisher Scientific, catalog number—11668019) according to the manufacturer's instructions to control for transfection efficiency. Transfected cells were incubated for 48 h and then treated with 2-DG for 6 h. Thereafter, luciferase activity was measured using dual luciferase assay kit (Promega) and bioluminometer (MDS Analytical Technologies). Final luciferase activity was calculated by normalizing firefly luciferase activity with Renilla luciferase activity and then the valued were converted to fold change with respect to control.

### Quantitative Real Time PCR

Total RNA was extracted from primary cortical neurons using NucleoSpin RNA II Kit (Clontech, Catalog number—740955-250). Thereafter, using the Taqman RNA-to-Ct, one step kit (Thermo Fisher Scientific, catalog number—4392938) and following the manufacturer's instructions, 50 nM of RNA from each sample was mixed with the Taqman® Gene Expression Master Mix and Taqman® Gene Expression Assays for Creb1 (Catalog number—Mm00501607_m1), Bdnf (Catalog number—Mm04230607_s1), Rbbp4 (Catalog number—Mm00771401_g1), Bip (Catalog number—Mm00517691_m1), Trib3 (Catalog number—Mm00454879_m1), and Chac1 (Catalog number—Mm00509926_m1) with FAM labelled probe and Actin with VIC labelled probe all from Thermo Fisher Scientific on an Applied Biosystems 7500 Fast Real Time PCR System. For assessing gene expression of various specific Bdnf exons in mouse primary neurons, two step qPCR was used. Total RNA from primary cortical neurons was extracted using NucleoSpin RNA II Kit (Clontech, Catalog number—740955-250) and a total of 1.5 μg RNA was used to synthesize cDNA with the SuperScript® III First-Strand Synthesis System (Thermo Fisher Scientific, Catalog number—18080051). Quantitative RT-PCR was then performed using SYBR green (Thermo Fisher Scientific, Catalog number—4309155) on an Applied Biosystems 7500 Fast Real Time PCR System. Quantitative levels for all genes were normalized to the housekeeping gene GAPDH and expressed relative to the relevant control samples as fold change. The sequences of primers used in this part of the study were as follows: Bdnf I forward: TTGAAGCTTTGCGGATATTGCG (SEQ ID NO:9) and Bdnf I reverse: AAGTTGCCTTGTCCGTGGAC (SEQ ID NO:10); Bdnf II forward: TGGTATACTGGGTTAACTTTGGGAAA (SEQ ID NO:11) and Bdnf II reverse: AAGTTGCCTTGTCCGTGGAC (SEQ ID NO:12); Bdnf IV forward #2: GAAATATATAGTAAGAGTCTAGAACCTTG (SEQ ID NO:13) and Bdnf IV reverse #2: AAGTTGCCTTGTCCGTGGAC (SEQ ID NO:14); Bdnf VI forward: GCTTTGTGTGGACCCTGAGTTC (SEQ ID NO:15) and Bdnf VI reverse: AAGTTGCCTTGTCCGTGGAC (SEQ ID NO:16); Bdnf VII forward: CCTGAAAGGGTCTGCGGAACTCCA (SEQ ID NO:17) and Bdnf VII reverse: GAAGTGTACAAGTCCGCGTCCTTA (SEQ ID NO:18) Bdnf IX forward: GGACTATGCTGCTGACTTGAAAGGA (SEQ ID NO:19) and Bdnf IX reverse: GAGTAAACGGTTTCTAAGCAAGTG (SEQ ID NO:20); Gapdh forward #2: ATGGTGAAGGTCGGTGTGAACG (SEQ ID NO:21) and Gapdh reverse #2: CGCTCCTGGAAGATGGTGATGG (SEQ ID NO: 22).

The gene expression of Bndf exon IV, Creba and Crebb in human i.p.s. derived neurons were also assessed using two step qPCR. Total RNA from human i.p.s derived neurons was isolated using mirVana kit (Thermo Fisher Scientific) according to manufacturer's instructions. A total of 1 μg RNA was used to synthesize cDNA with the SuperScript® III First-Strand Synthesis System (Thermo Fisher Scientific). Quantitative RT-PCR was then performed using SYBR green (Applied Biosystems) and the StepOnePlus™ Real-Time PCR System (Applied Biosystems). Quantitative levels for all genes were normalized to the housekeeping gene GAPDH and expressed relative to the relevant control samples. The sequences of primers used in this part of the study were as follows: Bdnf IV forward: GTGAGGTTTGTGTGGACCCC (SEQ ID NO:1) and Bdnf IV reverse: ATTGGGCCGAACTTTCTGGT (SEQ ID NO:2); Creba forward: GGCTCCAGATTCCATGGTC (SEQ ID NO:3) and Creba reverse: GTGTTACGTGGGGGAGAGAA (SEQ ID NO:4), Crebb forward: GGCTCCAGATTCCATGGTC (SEQ ID NO:5) and Crebb reverse: GTGTTACGTGGGGGAGAGAA (SEQ ID NO:6), and Gapdh forward: TGGTCTCCTCTGACTTCAACAGCG (SEQ ID NO:7) and Gapdh reverse: AGGGGTCTACATGGCAACTGTGAG (SEQ ID NO: 8).

### Immunoblot Analysis

Whole cell proteins were extracted in Triton X-100 lysis buffer (1% Triton X-100, 1% SDS, 50 mM Tris-Cl, pH 7.4, 500 mM NaCl and 1 mM EDTA). Samples were boiled in Laemmli buffer and electrophoresed under reducing conditions on NuPAGER Novex 4-12% Bis-Tris Gel polyacrylamide gels (Invitrogen). Proteins were transferred to a nitrocellulose membrane (Bio-Rad) by electroblotting. Nonspecific binding was inhibited by incubation in Odyssey blocking buffer (LI-COR Biosciences). Antibodies against Glut3 (Abcam, Catalog no. AB41525, dilution 1:1000), actin (Sigma Aldrich, Catalog no. A5316, dilution 1:10000), BDNF (Santacruz Biotechnology, Catalog no. sc-546, dilution 1:10,000), tubulin (Sigma Aldrich, Catalog no. T8203, dilution 1:10000), p-AMPK (Cell Signaling Technology, Cat no. 2535L, dilution 1:1000), AMPK (Cell Signaling Technology, Cat no. 2532L, dilution 1:1000), GFP (Cell Signaling Technology, Cat no. 2555S, dilution 1:2000), elF2S1 (phospho S51) (Abcam, Catalog no. AB32157, dilution 1:500), and eIF2α (Cell Signaling Technology, Cat no. 9722, dilution 1:500) were diluted in odyssey blocking buffer and the membranes were incubated overnight at 4° C. Fluorophore-conjugated Odyssey IRDye-680 or IRDye-800 secondary antibody (LI-COR Biosciences, Catalog numbers 926-32211 and 926-68070) was used at 1:10,000 dilution followed by incubation for 1 hour at room temperature. Finally, proteins were detected using an Odyssey infrared imaging system (LI-COR Biosciences).

### RNA Sequencing and Analysis

Primary cortical neurons were treated with 2-DG (1 mM and 10 mM) for 6 h from three independent cultures and RNA was isolated from each sample using a NucleoSpin RNA II Kit (Clontech, Catalog number—740955-250). For each RNA sample, RNA quality was initially quantified using the RNA Integrity Number (RIN) on an Agilent Bioanalyzer (Agilent Genomics). RNA-sequencing was carried out for the RNAs with by the UCLA Neuroscience Genomics Core. In brief, cDNA was generated using Ovation® RNA-Seq System V2 (NuGEN) followed by the library preparation using Illumina's TruSeq Stranded RNA (100 ng)+RiboZero Gold. The libraries were pooled and sequenced to generate 75 bp paired end reads on HiSeq™4000 system (Illumina). Minimum of 57M reads were obtained. Reads were aligned to the mouse mm10 reference genome using the STAR (ver 2.4.0) spliced read aligner (Dobin et al., 2013). Uniquely aligned read percentage was 88.09=0.53%(SD). Various quality matrix was generated to use high quality data for the analysis. Read counts for mouse refSeq genes were generated by HT-seq 0.6.1 (Anders et al., 2015). Genes with at least 5 read events at least half of samples were permitted into the dataset, for a total of 15,787 genes. Raw counts were normalized by trimmed mean of M values (TMM). Differentially expressed genes (DEG) were analyzed using an EdgeR bioconductor R (Robinson et al., 2010). False discovery rate less than 0.1 is used to define differentially expressed genes. Gene Set Enrichment Analysis (GSEA) was performed with gene lists sorted by directional p-Values from differential expression analysis. Raw and processed RNAseq data are deposited to Gene Expression Omnibus (GEO #).

### Chromatin Immunoprecipitation Sequencing (ChIP-Seq)

ChIP-seq was performed according to the ChIPmentation protocol with minor modifications. In brief, 40 million primary neurons were used for each condition. Cells were fixed in methanol free formaldehyde (1% final concentration) for 10 min at room temperature with slow rotation to allow the cross-linking of chromatin proteins and DNA. Cross-linking was stopped by incubation with 0.125 M glycine for 5 min at room temperature. The cells were washed twice with ice-cold phosphate-buffered saline (PBS) and 2 ml of cell scrapping solution was added cell were scrapped and collected in respective tubes. Tubes with cells were centrifuged at 2800 rpm for 10 min at 4° C. and supernatant was aspirated out. Cell pellets left in the tube were resuspended in 300 μl of 0.25% SDS sonication buffer (10 mM Tris-HCl [pH 8.0], 2 mM EDTA, 0.25% SDS, and protease inhibitor cocktail). The lysates were transferred to 1.5 ml TPX microtubes for sonication (Diagenode, Catalog number: 20190430) and sonicated by Biorupter sonication device (Diagenode) to shear genomic DNA into 200-600 bp fragments. The lysates were centrifuged to remove debris and were then diluted 1:1.5 in equilibration buffer (10 mM Tris-HCl [pH 8.0], 1 mM EDTA, 1.67% Triton X-100, 0.17% sodium deoxycholate, 233 mM NaCl, and protease inhibitor cocktail). Samples were centrifuged again at 14,000×g, 4 ºC for 10 minutes to pellet insoluble material and supernatant was transferred to a new tube. 350 μl of RIPA-LS with added inhibitors was added to the chromatin samples. 600 μl of the chromatin sample was used as I.P. fraction and remaining 45 μl of the chromatin sample was used as input fraction and was preserved at −80° C. for use. 15 μg of ATF4 antibody (Millipore, catalog number: ABE387) was added to the I.P. fraction. Both I.P. fraction with added ATF4 antibody and washed Dynabead Protein G (washed with 0.1% BSA/RIPA-LS buffer) were incubated in parallel in separate tubes overnight at 4° ° C. on a rotator with slow rotation. Next day, dynabeads protein G was added to I.P. fraction tube (25 μl per sample) and the complex was incubated again at 4° C. on a rotator for 2 h with slow rotation. The immuno-complexes were washed twice for 3 min each at 4° C. on a rotator with slow rotation with the following buffers: RIPA-low-salt wash buffer (10 mM Tris-HCl [pH 8.0], 1 mM EDTA, 140 mM NaCl, 0.1% SDS, 0.1% sodium deoxycholate, and 1% Triton X-100), RIPA-high-salt wash buffer (10 mM Tris-HCl [pH 8.0], 1 mM EDTA, 500 mM NaCl, 0.1% SDS, 0.1% sodium deoxycholate, and 1% Triton X-100), RIPA-LiCl wash buffer (10 mM Tris-HCl [pH 8.0], 1 mM EDTA, 250 mM LiCl, 0.5% Nonidet P-40, and 0.5% sodium deoxycholate), and TE buffer (10 mM Tris-HCl [pH 8.0] and 1 mM EDTA). Input fraction was thawed on ice. The bead-bound immunoprecipitated DNA and input DNA were tagmented in 25 μl tagmentation reaction containing 5 μl of 5× tagmentation buffer (Illumina, catalog number: 20034210), 19 μl of nuclease free water, and 1 μl of Tn5 (Illumina, catalog number: 20034210) at 37° ° C. for 3 min. Tn5 transposase cleaves double-stranded DNA and ligate adaptors at both ends. Tn5 was inactivated by adding RIPA-LS to the tagmentation reaction and incubating the tube for 5 min on ice. The beads were washed again with RIPA-LS and TE buffer twice each for 3 min at 4ºC on a rotator with slow rotation. Beads were then resuspended in 48 μl of ChIP elution buffer (10 mM Tris-HCl [pH 8.0], 5 mM EDTA, 300 mM NaCl, 0.4% SDS, and 2 μl of Proteinase K (Thermofisher Scientific, catalog number: 26160) at room temperature and were incubated at 55° C. for 1 h, followed by 65° C. incubation for 6 h for reversing the cross-linking and eluting the tagmented DNA. The eluted DNA was purified following SPRI bead cleanup method using AMPureXP beads (Beckman Coulter, catalog number: A63880). To reverse the crosslinking of DNA in input fraction, SDS (0.4% final concentration), NaCl (300 mM final concentration), and 2 μl proteinase K were added into the input sample and the sample was incubated at 55° C. for 1 h, followed by 65° C. incubation for 6 h. To prepare ChIP and input libraries, tagmented immunoprecipitated DNA and input DNA were amplified by PCR, each with a unique index incorporated. Libraries were selected by size using AMPureXP beads (Beckman Coulter, catalog number: A63880). For high throughput sequencing, DNA libraries were generated using NEBNext® ChIP-Seq Library Prep Master Mix Set for Illumina (NEB) and sequenced using an Illumina Novaseq S1 2×100 bp to obtain an average depth of 50 million of reads per sample.

### ChIP-Seq Data Analysis

Raw sequencing fastq files were assessed for quality, adapter content and duplication rates with the FastQC, trimmed using trim-galore (https://github.com/FelixKrueger/TrimGalore) and aligned to mouse genome (mm10) with BWA—mem with default parameters (Li and Durbin, 2009). PCR duplicates were removed using Picard MarkDuplicates (https://github.com/broadinstitute/picard) and the bigWig files were created using deepTools (Ramirez et al., 2016) with following parameters: bamCompare—binSize 20—normalizeUsing RPKM. Normalized bigwig files were used to generate heatmaps to visualize sample correlations and to remove outliers. MACS2 (Zhang et al., 2008) was used to call narrow peaks with input control with the following command: macs2 callpeak -t [ChIP BAM]-c [Input BAM]-f BAMPE -g mm—min-length 100—q 0.05. We used DiffBind (Stark and Brown, 2011) to calculate differences in peak levels between samples. HOMER (Heinz et al., 2010) findMotifsGenome.pl script was used for motif enrichment analysis of differential binding peaks was done by DiffBind (FDR<0.05). Motif models were drawn from both HOMER and JASPAR database (Sandelin et al., 2004).

### Long Term Potentiation

Brains were quickly removed from mice sacrificed by cervical dislocation and placed in cold artificial cerebrospinal fluid (ACSF) (bubbled with 95% O2/5% CO2) containing (in mM): 124 NaCl, 4 KCl, 1 Na2HPO4, 25 NaHCO₃, 2 CaCl2, 2 MgCl2, and 10 glucose. The pH and osmolarity of the solution were 7.4 and 310 mOsm/L, respectively. The hippocampus was isolated and placed on a mechanical tissue chopper to produce transverse hippocampal slices of 400 μm thickness. Slices were maintained in a humidified interface chamber at 29° C. and continuously perfused (˜1 mL/min) with 95% O2/5% CO2-bubbled ACSF. Hippocampal slices were allowed to recover for at least 90 min prior to beginning the experiment. For recordings, glass electrodes were filled with ACSF and positioned in the CA1 stratum radiatum to record fEPSPs evoked by local stimulation (0.1 ms) of Schaffer collateral fibers using a bipolar concentric electrode placed laterally to the recording electrode (˜150 μm). For LTP recordings, the voltage intensity of the stimulation test pulse (square pulse, 100 us duration) for each slice was determined to be the voltage intensity that had generated 30-40% of the maximum slope obtained using input-output relationships. Facilitation was calculated as the ratio of the slopes of the second and first fEPSPs, and plotted as a function of the inter-pulse duration. For LTP, a test pulse was applied every minute. Following a steady baseline of 15 min, potentiation was induced with either 100 Hz for 1 s (weak stimulation) or 3 theta-burst stimulations (TBS; 15 s interval), each one involving a single train of 10 bursts at 5 Hz, where each burst is composed of 4 pulses at 100 Hz (strong stimulation). The fEPSP slopes following tetanic stimulation were normalized to the average of the slopes of the fEPSPs acquired during the baseline. Residual potentiation was calculated as the average of the last 15 min of 2 h of recordings.

### In-Vivo Drug and Viral Administration

### Stereotaxic Continuous Brain Infusion of 2-DG Through Alzet Mini-Osmotic Pump Implantation

The sterile Alzet mini osmotic pumps (Durect, pump model 1004) were filled with 100 μl of either saline or 2-deoxyglucose (10 μg/μl) and were incubated in saline for 24 h to allow the osmotic release of the drug. Thereafter, the pumps were attached properly with the plastic tubing provided in the brain infusion kit 2 3-5 mm (Durect, catalog number 0008663). We made an intracerebroventricular groove stereotaxically in the mouse brain. We positioned the pump under the skin at the base of the neck and pushed it back toward the left hind limb as far as it went without resistance. We made sure to not let the catheter touch anything. With the curved hemostat, we fixed the cannula at the groove where the top meets the pedestal. We moved the cannula driver into position and secured into place. Then, we used metabond quick adhesive cement system (contains liquid dentin) to seal the cannula on the surface of the skull so that cannula stays there properly for 4-6 weeks. We pushed the top of the cannula into the driver at proper position so that the tubing is pointed straight back. We drove the thin metal catheter through the skull until the plastic cannula base is securely pressed against the top of the skull. The metal catheter can be driven directly through the skull in mice due to the relative thin skull. We pulled any skin that had glue on it away from the skull. With the cannula driver holding the cannula/catheter in place, we waited 1-2 min for the glue to fully dry. We held the catheter in place with the curved hemostat while raising the driver. Then, we slowly released the hemostat to ensure that the cannula is properly secured to the skull. With a cotton swab, we pressed down on the top of the cannula. We fitted the clippers into the grove between the top and base of the cannula and clipped off the top of the cannula while still pressing down with the cotton swab. We kept the clippers level so as not to detach the cannula from the skull. If the cannula came unglued, we quickly re-glued and applied pressure with a cotton swab for an additional 2 min. We closed the opening with clipper and added antibiotic ointment over the head and neck. Thereafter, we unscrewed the ear bars, loosened the nose cone and removed the mouse from the stereotaxic platform and placed on a warming pad for recovery. We monitored the mice closely for the duration of recovery, typically ranging from 10-30 minutes. We checked every 2-3 minutes until the mouse begins walking around and grooming itself. To control post-operative pain, meloxicam (1-2 mg/kg) was administered subcutaneously, and repeated doses were only administered based on presentation of discomfort/stress in the animals, including hunching, piloerection, vocalization, poor feeding and/or hydration. Animals were monitored daily for the sign of infection at the incision site. We kept the mice back in their respective cages with their proper food and water with 12 h light and dark cycle for four weeks and then proceeded with experiments such as learning and memory related behavior study, LTP study and gene expression studies.

### Stereotaxic Intracerebroventricular Administration of 2-DG

Using a nanomite syringe pump and Hamilton syringe, 5 μl of either saline or 2-DG (1 μg/μl) was infused directly into the ventricles at a rate of 0.120 ml/min in mice, which were injected with AAV8-CMV-5′UTR ATF4 luciferase intracranial double injection three weeks before. The injection site relative to the bregma point was lateral, 0.05; anteroposterior, 0.12 and dorsoventral, 0.25. Surgeon was blinded to treatment and control groups.

### Intraperitoneal Injection of 2-DG

To assess the effect of 2-DG on the Bdnf gene expression, mice were injected with either saline or 2-DG (10 mg/Kg) intraperitoneally for 6 h and then mice were euthanized properly, and hippocampi were dissected out from mice brain. In order to assess functional improvement with 2-DG treatment after ischemic stroke, mice were injected with 2-DG (10 mg/Kg) intraperitoneally 24 h after induction of the stroke and then every day for four weeks. For assessing functional improvement with 2-DG treatment after hemorrhagic stroke, mice were injected with 2-DG (10 mg/Kg) intraperitoneally 24 h after induction of the stroke and then every day for three weeks.

### Collagenase-Induced Intracerebral Hemorrhage (ICH) in Mouse

ICH was induced as described before (Karuppagounder et al., 2016). Briefly, male C57BL/6 mice (8 to 10 weeks of age; Charles River) were anesthetized with isoflurane (2 to 5%) and placed on a stereotaxic frame. During the procedure, the animal's body temperature was maintained at 37° ° C. with a homeothermic blanket. 1 ml of collagenase (0.075 IU; Sigma) was infused into the right striatum at a flow rate of 0.120 ml/min with a nanomite syringe pump (Harvard Apparatus) and a Hamilton syringe. The stereotaxic coordinates of the injection relative to the bregma point were as follows: lateral, −0.20; anteroposterior, 0.62; and dorsoventral, −0.40. 1 ml of saline was infused in control animals. The animals were randomized to sham or ICH groups. The data was collected in a blinded fashion and the identity was revealed after collection of the data.

### Filament MCAO Model of Ischemic Stroke in Mouse

The ischemic stroke was induced using filament MCAO method as described before (Alim et al., 2019). In brief, all surgeries were conducted in sterile conditions. Male mice were anesthetized with isoflurane (5% induction, and 2% maintenance). A 2 cm incision was opened in the middle of the anterior neck. The right common carotid was temporarily ligated with 6-0 silk (Ethicon Inc.). Right unilateral MCAO was accomplished by inserting a Silicon rubber-coated monofilament (Doccol Corporation) into the internal carotid artery via the external carotid artery stump. Adequacy of MCAO was confirmed by monitoring cortical blood flow at the onset of the occlusion with a laser Doppler flowmetry probe affixed to the skull (Periflux System 5010; Perimed, Sweden). Animals were excluded if mean intra-ischemic laser Doppler flowmetry was >30% pre-ischemic baseline. Transient focal cerebral ischemia was induced in mice for 60 minutes by reversible MCAO in the right brain hemisphere under isoflurane anesthesia followed by 24 hours of reperfusion. Body temperature was controlled at 36.5=0.5_C throughout MCAO surgery with warm water pads and a heating lamp. After 60 minutes of occlusion, the occluding filament was withdrawn to allow for reperfusion and the incision was closed with 6-0 surgical sutures (ETHICON, Inc). After surgery, 0.5 ml pre-warmed normal saline was given subcutaneously to each mouse. Mice were then allowed to recover from anesthesia and were survived for 24 h after initiation of the reperfusion.

### In Vivo Bioluminescence Imaging

In order to assess the induction of ATF4 in response to 2-DG treatment, we injected AAV8 viral vector expressing CMV-5′UTR-ATF4 tagged with luciferase through intracranial double injection and allowed maximal expression for three weeks and then injected either saline or 2-DG (1 mg/Kg) intracerebroventricularly for 4 h. Thereafter, mice were placed in the In Vivo Imaging System (IVIS; PerkinElmer) induction chamber and anesthetized with isoflurane (3 to 4% with an oxygen flow of 1 liter/min). The mice were individually removed from the induction chamber and given an intraperitoneal injection of luciferin (150 mg/kg; Promega) suspended in sterile saline (Invitrogen). After a 10-min incubation period, the mice were placed on the imaging platform of the IVIS Spectrum imaging station supplied with isoflurane at 1.5% with an oxygen flow of 1 liter/min during the imaging procedure. White light and luciferase activity images were obtained at 30-s intervals for 5 min. After imaging, the mice were removed from the imaging stage and were allowed to recover in a heated cage. Images were analyzed to quantify luminescence in either the brain or liver using Living Image software (PerkinElmer).

### Behavioral Analysis

### Corner Turn Test

The integrated sensorimotor function in both stimulation of vibrissae (sensory neglect) and rearing (motor response) was assessed through the corner turn test as described previously (Karuppagounder et al., 2016). Mice were placed between two cardboard pieces forming a corner with a 30° angle. While maintaining the 30° angle, the boards were gradually moved toward the mouse until the mouse approached the corner, reared upward, and turned 180° to face the open end. The direction (left or right) in which the mouse turned around was recorded for each trial. Ten trials were performed for each mouse.

### Adhesive Tape Removal Task

The adhesive tape removal task in mice was performed as previously described (Karuppagounder et al., 2016). Briefly, adhesive tape was placed on the planter region of the forward paw (right and left) of mice. The time from which the tape was applied to when the mouse successfully removed it was recorded for each paw. A maximum of 300 s for each paw was allowed.

### Pole Test

The Pole test assesses motor function. Pole test as performed as previously described (Balkaya et al., 2013). Animals were placed on top of a 50- to 55-cm vertical pole with a diameter of 8 to 10 mm and were trained to descend the pole with their snouts facing downward. Scoring started when the animal initiated the turning movement. The latency to reach the ground were recorded. However, if an animal fell immediately or stopped descending, the trial was excluded and repeated. The surface of the pole was made rough with adhesive tape to avoid sliding.

### Spontaneous Alternation Test (Y-Maze Test)

Short term spatial memory was assessed by testing spontaneous alteration behavior in the Y-maze. Mouse prefers to explore a new arm of the Y-maze instead of coming back to the previous arm, which was already visited. Y-maze has three equal arms each spaced at 120 degrees with respect to other arms. Recording of the testing began with the release of mouse in one arm and the mouse was allowed to explore in different arms of the maze for 8 min. The sequence and the total number of arm entries were recorded. The mouse was considered to be within one arm when paws of the mouse were completely in that arm. An alternation was considered complete when mouse entered in all three arms in a consecutive manner. The number of total alternation was calculated as the total number of arm entries minus 2 and the percentage of alternation was calculated as (actual alternation/total number of entries)×100.

### Morris Water Maze Test

Spatial learning and memory were analyzed using the Morris water maze. The mice were handled daily, starting 1 week before behavioral testing, to habituate them. During the acquisition period, visual cues were arranged in the four corners of the tank. The hidden platform was located in the middle of the northwest quadrant. Each day, mice were placed next to and facing the wall of the basin in four starting positions: north, east, south, and west, corresponding to four successive trials per day. The duration of a trial was 90 sec. Whenever the mouse failed to reach the platform within 90 s, it was placed on the platform by the experimenter for 10 sec. Latencies before reaching the platform were recorded for 7 days and analyzed. A probe trial was assessed 24 h after the last trial of the acquisition period by removing the platform from the pool. Mice were released on the north side for a single trial of 90 sec, during which the time spent in the area of the platform was measured. Latencies before reaching the platform were recorded and averaged.

### Statistical Analysis:

All experiments were performed as at least three independent sets and data were presented as means±standard deviation (SD). Statistical significances were assessed using GraphPad Prism using either Student's t tests to compare values between two specific groups or one-way ANOVA followed by Dunnett's post-hoc test/Tukey's Post-hoc test to compare the values of more than two groups or Repeated Measures two-way ANOVA with Tukey's multi-comparison test to compare the values of two or more than two groups at different time points. Statistical details for each figure can be found in the respective figure legend. The p value of 0.05 or less was considered statistically significant in all statistical analyses.

### REFERENCES

Alberini, C. M. (2008). The role of protein synthesis during the labile phases of memory: revisiting the skepticism. Neurobiol Learn Mem 89, 234-246.
Alim, I., Caulfield, J. T., Chen, Y., Swarup, V., Geschwind, D. H., Ivanova, E., Seravalli, J., Ai, Y., Sansing, L. H., Ste Marie, E. J., et al. (2019). Selenium Drives a Transcriptional Adaptive Program to Block Ferroptosis and Treat Stroke. Cell 177, 1262-1279 e1225.
Anders, S., Pyl, P. T., and Huber, W. (2015). HTSeq—a Python framework to work with high-throughput sequencing data. Bioinformatics 37, 166-169.
Axten, J. M., Medina, J. R., Feng, Y., Shu, A., Romeril, S. P., Grant, S. W., Li, W. H., Heerding, D. A., Minthorn, E., Mencken, T., et al. (2012). Discovery of 7-methyl-5-(1-{[3-(trifluoromethyl)phenyl]acetyl}-2,3-dihydro-1H-indol-5-yl)-7H-pyrrolo[2,3-d]pyrimidin-4-amine (GSK2606414), a potent and selective first-in-class inhibitor of protein kinase R (PKR)-like endoplasmic reticulum kinase (PERK). J Med Chem 55, 7193-7207.
Balkaya, M., Krober, J. M., Rex, A., and Endres, M. (2013). Assessing post-stroke behavior in mouse models of focal ischemia. J Cereb Blood Flow Metab 33, 330-338.
Balsa, E., Soustek, M. S., Thomas, A., Cogliati, S., Garcia-Poyatos, C., Martin-Garcia, E., Jedrychowski, M., Gygi, S. P., Enriquez, J. A., and Puigserver, P. (2019). ER and Nutrient Stress Promote Assembly of Respiratory Chain Supercomplexes through the PERK-cIF2alpha Axis. Mol Cell 74, 877-890 e876.
Chen, W. W., Freinkman, E., Wang, T., Birsoy, K., and Sabatini, D. M. (2016). Absolute Quantification of Matrix Metabolites Reveals the Dynamics of Mitochondrial Metabolism. Cell 166, 1324-1337 e1311.
Ciobanu, O., Elena Sandu, R., Tudor Balscanu, A., Zavaleanu, A., Gresita, A., Petcu, E. B., Uzoni, A., and Popa-Wagner, A. (2017). Caloric restriction stabilizes body weight and accelerates behavioral recovery in aged rats after focal ischemia. Aging Cell 16, 1394-1403.
Costa-Mattioli, M., Gobert, D., Harding, H., Herdy, B., Azzi, M., Bruno, M., Bidinosti, M., Ben Mamou, C., Marcinkiewicz, E., Yoshida, M., et al. (2005). Translational control of hippocampal synaptic plasticity and memory by the elF2alpha kinase GCN2. Nature 436, 1166-1173.
Costa-Mattioli, M., Gobert, D., Stern, E., Gamache, K., Colina, R., Cuello, C., Sossin, W., Kaufman, R., Pelletier, J., Rosenblum, K., et al. (2007). cIF2alpha phosphorylation bidirectionally regulates the switch from short- to long-term synaptic plasticity and memory. Cell 129, 195-206.
de Cabo, R., and Mattson, M. P. (2019). Effects of Intermittent Fasting on Health, Aging, and Disease. N Engl J Med 381, 2541-2551.
Dias, I. R., Santos, C. S., Magalhaes, C., de Oliveira, L. R. S., Peixoto, M. F. D., De Sousa, R. A. L., and Cassilhas, R. C. (2020). Does calorie restriction improve cognition? IBRO Rep 9, 37-45.
Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21.
Duan, W., and Mattson, M. P. (1999). Dietary restriction and 2-deoxyglucose administration improve behavioral outcome and reduce degeneration of dopaminergic neurons in models of Parkinson's disease. J Neurosci Res 57, 195-206.
Frakes, A. E., and Dillin, A. (2017). The UPR(ER): Sensor and Coordinator of Organismal Homeostasis. Mol Cell 66, 761-771.
Fujii, Y., Shimizu, T., Toda, T., Yanagida, M., and Hakoshima, T. (2000). Structural basis for the diversity of DNA recognition by bZIP transcription factors. Nat Struct Biol 7, 889-893.
Garriga-Canut, M., Schoenike, B., Qazi, R., Bergendahl, K., Daley, T. J., Pfender, R. M., Morrison, J. F., Ockuly, J., Stafstrom, C., Sutula, T., et al. (2006). 2-Deoxy-D-glucose reduces epilepsy progression by NRSF-CtBP-dependent metabolic regulation of chromatin structure. Nat Neurosci 9, 1382-1387.
Grontvedt, G. R., Schroder, T. N., Sando, S. B., White, L., Brathen, G., and Doeller, C. F. (2018). Alzheimer's disease. Curr Biol 28, R645-R649.
Gudden, J., Arias Vasquez, A., and Bloemendaal, M. (2021). The Effects of Intermittent Fasting on Brain and Cognitive Function. Nutrients 13.
Halagappa, V. K., Guo, Z., Pearson, M., Matsuoka, Y., Cutler, R. G., Laferla, F. M., and Mattson, M. P. (2007). Intermittent fasting and caloric restriction ameliorate age-related behavioral deficits in the triple-transgenic mouse model of Alzheimer's disease. Neurobiol Dis 26, 212-220.
Heinz, S., Benner, C., Spann, N., Bertolino, E., Lin, Y. C., Laslo, P., Cheng, J. X., Murre, C., Singh, H., and Glass, C. K. (2010). Simple combinations of lineage-determining transcription factors prime cis-regulatory elements required for macrophage and B cell identities. Mol Cell 38, 576-589.
Horie, N.C., Serrao, V. T., Simon, S. S., Gascon, M. R., Dos Santos, A. X., Zambone, M. A., Del Bigio de Freitas, M. M., Cunha-Neto, E., Marques, E. L., Halpern, A., et al. (2016). Cognitive Effects of Intentional Weight Loss in Elderly Obese Individuals With Mild Cognitive Impairment. J Clin Endocrinol Metab 101, 1104-1112.
Huang, J. T., Mao, Y. Q., Han, B., Zhang, Z. Y., Chen, H. L., Li, Z. M., Kong, C. Y., Xu, J. Q., Cai, P. R., Zeng, Y. P., et al. (2021). Calorie restriction conferred improvement effect on long-term rehabilitation of ischemic stroke via gut microbiota. Pharmacol Res 170, 105726.
Jiang, Z., Belforte, J. E., Lu, Y., Yabe, Y., Pickel, J., Smith, C. B., Je, H. S., Lu, B., and Nakazawa, K. (2010). cIF2alpha Phosphorylation-dependent translation in CA1 pyramidal cells impairs hippocampal memory consolidation without affecting general translation. J Neurosci 30, 2582-2594.
Karuppagounder, S. S., Alim, I., Khim, S. J., Bourassa, M. W., Sleiman, S. F., John, R., Thinnes, C. C., Ych, T. L., Demetriades, M., Neitemeier, S., et al. (2016). Therapeutic targeting of oxygen-sensing prolyl hydroxylases abrogates ATF4-dependent neuronal death and improves outcomes after brain hemorrhage in several rodent models. Sci Transl Med 8, 328ra329.
Kishi, T., Hirooka, Y., Nagayama, T., Isegawa, K., Katsuki, M., Takesuc, K., and Sunagawa, K. (2015). Calorie restriction improves cognitive decline via up-regulation of brain-derived neurotrophic factor: tropomyosin-related kinase B in hippocampus ofobesity-induced hypertensive rats. Int Heart J 56, 110-115.
Kowianski, P., Lictzau, G., Czuba, E., Waskow, M., Steliga, A., and Morys, J. (2018). BDNF: A Key Factor with Multipotent Impact on Brain Signaling and Synaptic Plasticity. Cell Mol Neurobiol 38, 579-593.
Kumar, A. (2019). Sugar as a therapeutic target for the cognitive restoration following traumatic brain injury. Curr Opin Neurol 32, 815-821.
Lane, M. A., Ingram, D. K., and Roth, G. S. (1998). 2-Deoxy-D-Glucose feeding in rats mimics physiologic Eeffects of calorie restriction. Journal of Anti-Aging Medicine 1, 327-337.
Leal, G., Bramham, C. R., and Duarte, C. B. (2017). BDNF and Hippocampal Synaptic Plasticity. Vitam Horm 104, 153-195.
Leclerc, E., Trevizol, A. P., Grigolon, R. B., Subramaniapillai, M., McIntyre, R. S., Brictzke, E., and Mansur, R. B. (2020). The effect of caloric restriction on working memory in healthy non-obese adults. CNS Spectr 25, 2-8.
Lec, J., Duan, W., Long, J. M., Ingram, D. K., and Mattson, M. P. (2000). Dictary restriction increases the number of newly generated neural cells, and induces BDNF expression, in the dentate gyrus of rats. J Mol Neurosci 15, 99-108.
Li, H., and Durbin, R. (2009). Fast and accurate short read alignment with Burrows-Wheeler transform. Bioinformatics 25, 1754-1760.
Liu, Y., Cheng, A., Li, Y. J., Yang, Y., Kishimoto, Y., Zhang, S., Wang, Y., Wan, R., Racfsky, S. M., Lu, D., et al. (2019). SIRT3 mediates hippocampal synaptic adaptations to intermittent fasting and ameliorates deficits in APP mutant mice. Nat Commun 10, 1886.
Ma, T., Trinh, M. A., Wexler, A. J., Bourbon, C., Gatti, E., Pierre, P., Cavener, D. R., and Klann, E. (2013). Suppression of cIF2alpha kinases alleviates Alzheimer's disease-related plasticity and memory deficits. Nat Neurosci 16, 1299-1305.
Mattson, M. P., Mochl, K., Ghena, N., Schmacdick, M., and Cheng, A. (2018). Intermittent metabolic switching, neuroplasticity and brain health. Nat Rev Neurosci 19, 63-80.
Nair, A. B., and Jacob, S. (2016). A simple practice guide for dose conversion between animals and human. J Basic Clin Pharm 7, 27-31.
Ooi, T. C., Meramat, A., Rajab, N. F., Shahar, S., Ismail, I. S., Azam, A. A., and Sharif, R. (2020). Intermittent Fasting Enhanced the Cognitive Function in Older Adults with Mild Cognitive Impairment by Inducing Biochemical and Metabolic changes: A 3-Year Progressive Study. Nutrients 12.
Ota, M., Matsuo, J., Ishida, I., Takano, H., Yokoi, Y., Hori, H., Yoshida, S., Ashida, K., Nakamura, K., Takahashi, T., et al. (2019). Effects of a medium-chain triglyceride-based ketogenic formula on cognitive function in patients with mild-to-moderate Alzheimer's disease. Neurosci Lett 690, 232-236.
Pasini, S., Corona, C., Liu, J., Greene, L. A., and Shelanski, M. L. (2015). Specific downregulation of hippocampal ATF4 reveals a necessary role in synaptic plasticity and memory. Cell Rep 11, 183-191.
Racz, L. E., Papadopoulos, K., Ricart, A. D., Chiorcan, E. G., Dipaola, R. S., Stein, M. N., Rocha Lima, C. M., Schlesselman, J. J., Tolba, K., Langmuir, V. K., et al. (2013). A phase I dose-escalation trial of 2-deoxy-D-glucose alone or combined with docetaxel in patients with advanced solid tumors. Cancer Chemother Pharmacol 71, 523-530.
Ramirez, F., Ryan, D. P., Gruning, B., Bhardwaj, V., Kilpert, F., Richter, A. S., Heyne, S., Dundar, F., and Manke, T. (2016). deepTools2: a next generation web server for deep-sequencing data analysis. Nucleic Acids Res 44, W160-165.
Ratan, R. R., Murphy, T. H., and Baraban, J. M. (1994). Macromolecular synthesis inhibitors prevent oxidative stress-induced apoptosis in embryonic cortical neurons by shunting cysteine from protein synthesis to glutathione. J Neurosci 14, 4385-4392.
Rink, C., Gnyawali, S., Stewart, R., Teplitsky, S., Harris, H., Roy, S., Sen, C. K., and Khanna, S. (2017). Glutamate oxaloacetate transaminase enables anaplerotic refilling of TCA cycle intermediates in stroke-affected brain. FASEB J 31, 1709-1718.
Roberge, M. C., Messier, C., Staines, W. A., and Plamondon, H. (2008). Food restriction induces long-lasting recovery of spatial memory deficits following global ischemia in delayed matching and non-matching-to-sample radial arm maze tasks. Neuroscience 156, 11-29.
Robinson, M. D., Mccarthy, D. J., and Smyth, G. K. (2010). edgeR: a Bioconductor package for differential expression analysis of digital gene expression data. Bioinformatics 26, 139-140.
Rubovitch, V., Pharayra, A., Har-Even, M., Dvir, O., Mattson, M. P., and Pick, C. G. (2019). Dietary Energy Restriction Ameliorates Cognitive Impairment in a Mouse Model of Traumatic Brain Injury. J Mol Neurosci 67, 613-621.
Sandelin, A., Alkema, W., Engstrom, P., Wasserman, W. W., and Lenhard, B. (2004). JASPAR: an open-access database for cukaryotic transcription factor binding profiles. Nucleic Acids Res 32, D91-94.
Santini, E., Huynh, T. N., and Klann, E. (2014). Mechanisms of translation control underlying long-lasting synaptic plasticity and the consolidation of long-term memory. Prog Mol Biol Transl Sci 122, 131-167.
Sidrauski, C., Acosta-Alvear, D., Khoutorsky, A., Vedantham, P., Hearn, B. R., Li, H., Gamache, K., Gallagher, C. M., Ang, K. K., Wilson, C., et al. (2013). Pharmacological brake-release of mRNA translation enhances cognitive memory. Elife 2, c00498.
Smith, P. J., Blumenthal, J. A., Babyak, M. A., Craighead, L., Welsh-Bohmer, K. A., Browndyke, J. N., Strauman, T. A., and Sherwood, A. (2010). Effects of the dietary approaches to stop hypertension diet, exercise, and caloric restriction on neurocognition in overweight adults with high blood pressure. Hypertension 55, 1331-1338.
So, J. S., Cho, S., Min, S. H., Kimball, S. R., and Lec, A. H. (2015). IREIalpha-Dependent Decay of CReP/Ppp1r15b mRNA Increases Eukaryotic Initiation Factor 2alpha Phosphorylation and Suppresses Protein Synthesis. Mol Cell Biol 35, 2761-2770.
Stark, R., and Brown, G. (2011). DiffBind: Differential binding analysis of ChIPSeq peak data. Bioconductor.
Stein, M., Lin, H., Jeyamohan, C., Dvorzhinski, D., Gounder, M., Bray, K., Eddy, S., Goodin, S., White, E., and Dipaola, R. S. (2010). Targeting tumor metabolism with 2-deoxyglucose in patients with castrate-resistant prostate cancer and advanced malignancies. Prostate 70, 1388-1394.
Stuart, C. A., Ross, I. R., Howell, M. E., McCurry, M. P., Wood, T. G., Ceci, J. D., Kennel, S. J., and Wall, J. (2011). Brain glucose transporter (Glut3) haploinsufficiency does not impair mouse brain glucose uptake. Brain Res 1384, 15-22.
Sun, X., Aime, P., Dai, D., Ramalingam, N., Crary, J. F., Burke, R. E., Greene, L. A., and Levy, O. A. (2018). Guanabenz promotes neuronal survival via enhancement of ATF4 and parkin expression in models of Parkinson disease. Exp Neurol 303, 95-107.
Surani, M. A. (1979). Glycoprotein synthesis and inhibition of glycosylation by tunicamycin in preimplantation mouse embryos: compaction and trophoblast adhesion. Cell 18, 217-227.
Trinh, M. A., Kaphzan, H., Wek, R. C., Pierre, P., Cavener, D. R., and Klann, E. (2012). Brain-specific disruption of the eIF2alpha kinase PERK decreases ATF4 expression and impairs behavioral flexibility. Cell Rep 1, 676-688.
Wan, R., Camandola, S., and Mattson, M. P. (2004). Dietary supplementation with 2-deoxy-D-glucose improves cardiovascular and neuroendocrine stress adaptation in rats. Am J Physiol Heart Circ Physiol 287, H1186-1193.
Wang, Y. C., Li, X., Shen, Y., Lyu, J., Sheng, H., Paschen, W., and Yang, W. (2020). PERK (Protein Kinase RNA-Like ER Kinase) Branch of the Unfolded Protein Response Confers Neuroprotection in Ischemic Stroke by Suppressing Protein Synthesis. Stroke 51, 1570-1577.
Witte, A. V., Fobker, M., Gellner, R., Knecht, S., and Floel, A. (2009). Caloric restriction improves memory in elderly humans. Proc Natl Acad Sci USA 106, 1255-1260.
Xia, J., Sinelnikov, I. V., Han, B., and Wishart, D. S. (2015). MetaboAnalyst 3.0—making metabolomics more meaningful. Nucleic Acids Res 43, W251-257.
Yao, J., Chen, S., Mao, Z., Cadenas, E., and Brinton, R. D. (2011). 2-Deoxy-D-glucose treatment induces ketogenesis, sustains mitochondrial function, and reduces pathology in female mouse model of Alzheimer's disease. PLOS One 6, e21788.
Yu, Z. F., and Mattson, M. P. (1999). Dietary restriction and 2-deoxyglucose administration reduce focal ischemic brain damage and improve behavioral outcome: evidence for a preconditioning mechanism. J Neurosci Res 57, 830-839.
Zaghmi, A., Dopico-Lopez, A., Perez-Mato, M., Iglesias-Rey, R., Hervella, P., Greschner, A. A., Bugallo-Casal, A., da Silva, A., Gutierrez-Fernandez, M., Castillo, J., et al. (2020). Sustained blood glutamate scavenging enhances protection in ischemic stroke. Commun Biol 3, 729.
Zhang, Y., Liu, T., Meyer, C. A., Eeckhoute, J., Johnson, D. S., Bernstein, B. E., Nusbaum, C., Myers, R. M., Brown, M., Li, W., et al. (2008). Model-based analysis of ChIP-Seq (MACS). Genome Biol 9, R137.

1. A method of treating a neurodegenerative disease in a subject comprising administering directly into the central nervous system (CNS) of the subject an amount of an inhibitor of N-linked glycosylation thereby treating the neurodegenerative disease.

2. A method of protecting a cell in a subject against ferroptosis comprising administering to the subject an amount of an activator of an Integrated Stress Response pathway effective to increase phosphorylation of a Eukaryotic Initiation Factor 2 alpha (eif2alpha), or to elicit Bdnf gene expression, and thereby reduce ferroptosis, or a method of protecting a cell in a subject against ferroptosis, wherein the subject has, or is experiencing, a neurodegenerative disease comprising administering to the subject an amount of an activator of an Integrated Stress Response pathway effective to increase phosphorylation of an eif2alpha, or to elicit Bdnf gene expression, and thereby reduce ferroptosis.

3. The method of claim 2, wherein the neurodegenerative disease is stroke, Parkinson's disease, Alzheimer's disease, epilepsy, or Huntington's disease.

4. A method of treating a subject for a stroke, Parkinson's disease, Alzheimer's disease, epilepsy, or Huntington's disease, comprising administering to the subject an amount of an activator of an Integrated Stress Response pathway effective to increase phosphorylation of an eif2alpha so as to treat the stroke, Parkinson's disease, Alzheimer's disease, epilepsy, or Huntington's disease.

5. The method of claim 4, wherein the method increases phosphorylation of an eif2alpha and reduces ferroptosis.

6. The method of claim 1, further comprising administering an anti-ferroptotic agent to the subject.

7. The method of claim 6, wherein the anti-ferroptotic agent is a system xc-inhibitor, a GPX4 inhibitor, or a compound that indirectly inhibitsGPX4 activity by GSH depletion.

8. The method of claim 6, wherein the anti-ferroptotic agent is rifampicin, promethazine, omeprazole, indole-3-carbinol, carvedilol, propranolol, estradiol, or a thyroid hormone.

9. The method of claim 1, wherein the method treats Alzheimer's disease.

10. The method of claim 1, wherein the subject has had, or is experiencing, a stroke and wherein the method enhances stroke recovery in the subject.

11. The method of claim 2, wherein the activator of Integrated Response Pathway is 2-deoxyglucose.

12. The method of claim 1, wherein the subject (i) does not have a cancer, (ii) has not been diagnosed with a cancer, and/or (iii) has not been treated for a cancer.

13. The method of claim 1, wherein the subject (i) is not on a calorie-restricted diet regime, (ii) has not been prescribed a calorie-restricted diet regime by a healthcare provider, and/or (iii) is treated for stroke recovery by being administered the activator and without being on a calorie-restricted diet regime.

14. The method of claim 2, wherein the activator of Integrated Response Pathway is administered at least daily for a period of at least a week subsequent to the subject having a stroke.

15. The method of claim 2, wherein the activator of Integrated Response Pathway is administered at least daily for a period of at least four weeks subsequent to the subject having a stroke.

16. The method of claim 10, wherein the activator of Integrated Response Pathway is 2-deoxyglucose and is administered to a human subject in an amount of at least 50 ug/Kg body weight/day.

17. The method of claim 10, wherein the activator of Integrated Response Pathway is 2-deoxyglucose and is administered to a human subject in an amount of at least 100 ug/Kg body weight/day.

18. The method of claim 10, wherein the activator of Integrated Response Pathway is 2-deoxyglucose and is administered to a human subject in an amount of at least 500 ug/Kg body weight/day.

19. The method of claim 10, wherein the activator of Integrated Response Pathway is 2-deoxyglucose and is administered to a human subject in an amount of at least 1 mg/Kg body weight/day.

20. The method of claim 1, wherein the administration of the treatment does not elicit hypoglycemia in the subject.

21-63. (canceled)
